SEQUENCE LISTING (47 sequences)

SEQ ID NO: 1 - DNA, 354 bp, Homo Sapien
  misc_feature 152, 157, 169: n = A,T,C or G
ccattctgagtacttctccgcaaaccctttgtttcattaaggactgttttacatgaagggtgcaaaagtaggataaaaatgagaaccctagggtgaaacacgtgacagaagaataaagactattgaatagtcctcttctctacccatggacnttggnatttttatattngattttaaggaaatataacttagtagtaaagagatgagcattcaagtcaggcagacctgaatttgggtcaaggctgcgccactcaaaagctatatgacctctatatgagcagcttattcaacctcttttaacctccattttgtcatctgtagaatgatgataaatgcctagctcagaaggattcc

SEQ ID NO: 2 - DNA, 543 bp, Homo Sapien
gttcaactttagagggagccatggggactaaacaaaattctgaggccccctcaaccatctaaatggacttccttctgggccaggacactcgaaaattaaacctgaaagactggttcaggccatgatgggaagtgggagtcgaacatgcctcatcataccctccagcattaacatcaacacagaccttaaggctgataagaagcatttacaatctattctctctgaagtcttctacctggaggcttcatctgcatgataaaactttggtctccacaacctcttacaacccaggcattcctttctatcgataattactctttcaaccaattgccaatcagaaaattgttatatctacctataatctagaagcccccacatcaagttgttttgcctttctggacaggaccaatgtatatcttaaatgtatttgattgatctctcatgtctccctaaaatgtataaaaccacgctgttccccgaccacctggagcacatgttctcagggtctcctgagggctgtgtcacaggccatgttcacttaca

SEQ ID NO: 3 - DNA, 513 bp, Homo Sapien
gacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctc

SEQ ID NO: 4 - DNA, 432 bp, Homo Sapien
gccacagactgaactcgcagggagtgcagcaggaaggaacaaagacaggcaaacggcaacgtagcctgggctcactgtgctggggcatggcgggatcctccacagagaggaggggaccaattctggacagacagatgttgggaggatacagaggagatgccacttctcactcaccactaccagccagcctccagaaggccccagagagaccctgcaagaccacggagggagccgacacttgaatgtagtaataggcagggggccctgccaccccatccagccagaccccagctgaaccatgcgtcaggggcctagaggtggagttcttagctatccttggctttctgtgccagcctggctctgcccctcccccatgggctgtgtcctaaggcccatttgagaagctgaggctagttccaaaaacctctcctg

SEQ ID NO: 5 - DNA, 546 bp, Homo Sapien
agccagttacaggtggagcagctcctgtacgaaagccctgagcggtactcccgctcagtgcttctcatcacccagcacctcagcctggtggagcaggctgaccacatcctctttctggaaggaggcgctatccgggaggggggaacccaccagcagctcatggagaaaaaggggtgctactgggccatggtgcaggctcctgcagatgctccagaatgaaagccttctcagacctgcgcactccatctccctcccttttcttctctctgtggtggagaaccacagctgcagagtagcagctgcctccaggatgagttacttgaaatttgccttgagtgtgttacctcctttccaagctcctcgtgataatgcagacttcctggagtacaaacacaggatttgtaattcctactgtaacggagtttagagccagggctgatgctttggtgtggccagcactctgaaactgagaaatgttcagaatgtacggaaagatgatcagctattttcaacataactgaaggcatatgctggcccataaacacc

SEQ ID NO: 6 - DNA, 502 bp, Homo Sapien
acaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaa

SEQ ID NO: 7 - DNA, 511 bp, Homo Sapien
tacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggcgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggagta

SEQ ID NO: 8 - DNA, 500 bp, Homo Sapien
gcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtccc

SEQ ID NO: 9 - DNA, 411 bp, Homo Sapien
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcacc

SEQ ID NO: 10 - DNA, 503 bp, Homo Sapien
gcggcatgtgaccatcattgaactggtgggacagccacctcaggaggtggggcgcatccgggagcaacagctgtcagccaacatcatcgaggagctcaggcaatttcagcgcctcactcgctcctacttcaacatggtgttgattgacaagcagggtattgaccgagaccgctacatggaacctgtcacccccgaggaaatcttcacattcattgatgactacctactgagcaatcaggagttgacccagcgtcgggagcaaagggacatatgcgagtgaacttgagccagggcatggttaaagtcaagggaaaagctcctctagttagctgaaactgggacctaataaaaggaggaaatgttttcccacagttctagggacaggactctgaggtgggtgagtttgacaaatcctgcagtgtttccaggcatccttttaggactgtgtaatagtttccctagaagctaggtagggactgaggacaggccttgggcagtgggtt

SEQ ID NO: 11 - DNA, 463 bp, Homo Sapien
agtcctctgcaagcacgtatgtgccaacggtgtgcaatgggcgggaagtcctcgactccaccacctcatctctgtgattgtgaattgcagttcagttcttgtgtttttagactgttagacaaaagtgctcacgtgcagctccagaatatggaaacagagcaaaagaacaaccctagtacctttttttagaaacagtacgataaattatttttgaggactgtatatagtgatgtgctagaactttctaggctgagtctagtgcccctattattaacaattcccccagaacatggaaataaccattgtttacagagctgagcattggtgacagggtctgacatggtcagtctactaaaaaacaaaaaaaaaaaacaaaaaaaaaaaaacaaaagaaaaaaataaaaatacggtggcaatattatgtaaccttttttcctatgaagttttttgtaggtccttgttg

SEQ ID NO: 12 - DNA, 508 bp, Homo Sapien
gatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctggggatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttga

SEQ ID NO: 13 - DNA, 468 bp, Homo Sapien
tgtccacaggcctgatccagaatggagactggaccttccagaccctggtgatgctggaaacagttcctcggagtggagaggtttacacctgccaagtggagcacccaagcctgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggtaataccttttcatcctctttaagaaacagatttggaggccaggcgcagtggctcacgcctgtaatcccagcactttgggaggccgaggcgggcgaatcatgaggtcaggagttcgagaccagcctgaccaacgtggtgaaaccccgtctctactaaaaatacaaaaaaaaatcagtcgggcgtggtggtgtgc

SEQ ID NO: 14 - DNA, 517 bp, Homo Sapien
gtggcggagcagctgagagcctacctggagggcgagtgcgtggagtggctccgcagatacctggagaacgggaaggagacgctgcagcgcgcggaccccccaaagacacacgtgacccaccaccccatctctgaccatgaggccaccctgaggtgctgggccctgggcttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagctcaggtggaaaaggagggagctactctcaggctg

SEQ ID NO: 15 - DNA, 425 bp, Homo Sapien
gctgtcctaggagctgtggtggctgttgttatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgcgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaactttacgtgtactgagctgcaacttcttacttc

SEQ ID NO: 16 - DNA, 530 bp, Homo Sapien
agactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataatggtggtggtggcagctgggcaggtctcctctgggaggtcttggccgactcagggacctaagcc

SEQ ID NO: 17 - DNA, 498 bp, Homo Sapien
  misc_feature 386: n = A,T,C or G
aatgggtttaatgaggtctaccttgcagagccattgtgagcattggaaatgatgaatgaatcataccagaacgtctagtataattacagtcatgcattgcttaacgatggggatacattcttagaaatgtgtcactaggcaattctgtcattgtgtaaacattataggatgtacttacacaaacctagatgttatatgtatttttatttacatgtatattttcacatgaaataccaaatgtcacagcattattactgaatgtcagtcatttcccctacttgatctgcaatgccaatatcaagggccatgtatcaggtttctgtatatgttccactataatcttatgggaccatggttttaaatgtggaatcattgacagaaatgtntttatgtagcatatggctgtgtatcactagtatataatagagcaatattatggaggaatatgtagatccaatcactttacctatacaaaatgactgctatggtgggaacaca

SEQ ID NO: 18 - DNA, 505 bp, Homo Sapien
ctgagagcctacctggagggcctgtgcgtggagtggctccgcagatacctggagaacgggaaggagacgctgcagcgcgcggaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatgggagccatcttcccagtccaccatccccatcgtgggcattgttgctggcctggctgtcctagcagttgtggtcatcggagctgtggtcgctactgtgatgtgtaggaggaagagctcaggtggaaaaggagggagctactctcaggctg

SEQ ID NO: 19 - DNA, 531 bp, Homo Sapien
  misc_feature 41, 43, 44, 114, 115, 116, 118, 119, 120, 121, 122, 123,124, 125, 127, 128, 129, 273, 337, 365, 396, 457, 460, 468: n = A,T,C or G
ccaacgcaaattcgcaaactttcttaaaacattatgagttncnntttgctatttttttttttttttttagctcatcggctatcgttagtgctagtggattttacatgtggcccnnnannnnnnnncnnncaacgtggcccagagaagccaaaagattggatacgcatcagacagatggaaaagggagattcagactgtttttcagggaggtggctgggtttacacgctaatcccgattcaccctgtccaaactgcctaagccctccgccattntcaagccctgcagtcacagctacacagatcacagcttcagccaggagctgggcagaaggccaanaggctgttcccaccaggctgctcagggntggtcttttaggacccttcccttgagccctntatggtgtggcaaagccttcattgccttaactggagccccatcagctccagctgctctgtnttntttgcccncaatgctttgcccctgagacaaatggaggcctgtcctgacctgtctcaccatgtacatagctt

SEQ ID NO: 20 - DNA, 553 bp, Homo sapien
  misc_feature 102, 199, 200, 202, 203, 205, 206, 207, 208, 209, 210,211, 212: n = A,T,C or G
cactactgctgcagctcacaaacacctctgcatattacatgtacctcctcctgctcctcaagagtgtggtctattttgccatcatcacctgctgtctgcttngaagaacggctttctgctgcaatggagagaaatcataacagacggtggcacaaggaggccatcttttcctcatcggttattgtccctagaagcgtcnncnnannnnnnnnttgggctttctttctgggtttgggccatttcagttctcatgtgtgtactattctatctattgtataatggttttcaaaccagtgggcacacagagaacctcactctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaata

SEQ ID NO: 21 - DNA, 526 bp, Homo Sapien
agtcctgtctgtgtggactggcacctgggctgctggagaagtctcctcccgttcggaccagcctcagggctgcacgttacctcaggaatgggccccaccatgaaggggcccatctgtcagcagcgtcttctaggtccccagctcagggagccatccccagctccagttttctcatgcgaatatgcacagttttaattcacgttgttacactagcctgccgatgagacccagacacaggcagacctggcgctcttgacccctgattccagtgaggactggccctgaggagtccttgcagacctgctgcctgccccacgacaggcccaaagatggaccccccctggccttgtgacagctccccaagtgttctccggtggagaaactgcagaggactggtgggcggggctccccagccatcaccatcctgtgtacaatggctgtagacttgtatatggctcctttaatattgtaaagatgctgatgtacaattgtcgtgcatttgtgtgaacacattgcgttcccagtc

SEQ ID NO: 22 - DNA, 470 bp, Homo Sapien
aaatcaggtcactcctttgcttacaacgcagtgcgtttagaggtacaccccatgtctccacagggcatacagcatccgatttaatctggatccattccggcgcttcctctcccagtcacccagagggccccaaccccggcggccctttcttcctcaaatgtcctcggctctataccgtgcctgggtcttttctctttctctctgcctggaacattccttctttccccttttgtcttgcccactcctgtttacccttcaagtttcaagttcatgtcactgtctcagagaggttttcctgtgctcgccctgtttctctcaggaagccttgctcttttccatcatgcctctaatcacagcttataatcggatatttatttctgtgtctacagtcttgccctgccagactgtaagccccatgtgggcaggcgctcatgattgtttctgattgtttcacgcatgctgctaaccca

SEQ ID NO: 23 - DNA, 429 bp, Homo Sapien
  misc_feature 129: n = A,T,C or G
tctaccctgcggagatcacgctgacctggcagcgggatggggaggaacagacccaggacacagagcttgtggagaccaggcctgcaggggatggaaccttccagaagtgggccgctgtggtggtgcctnctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacata

SEQ ID NO: 24 - DNA, 502 bp, Homo Sapien
ccctccccaacgttaaatgctcgagaggaatcttccacaaggcagggccatgcacgcaacctgcacacgcacttggagggcccaggtgtctctccaccagcccccatgcagtagggactggaagatatgtcatctgctggttgtgttatcactcccaccccctaccccagcccgtcttccggaatttctcaactaaatttcattattgggcaggaaggaggtcatgggttcatttcatttttgttttttgtgtttttaattaaaagaaaggttacctcagttttcactccttagacatggatgtagctacctttttttgtatgtctttttttttttaagcaatcgtgttgaattaggagtatacttggtgtggaaagagtatgaatttgccatgtgatttgcaaatggggggaagctactgtgagcgtgtgtttttttaatttacactatagagtgatttttttttcccccaacgtcaagtttttaccttgcatgtactgga

SEQ ID NO: 25 - DNA, 393 bp, Homo Sapien
aaactcctgttcagaaatggacttcagctgatcttcatattcatatggagtttccagtgaccccaaatagccaaaacagtcttggaaagaaaaacaaagttggaggacccacacttcctgattttgaaacttgctacaaagctatagtactcaacaaagattggtaatggcataaggatatagattaagaacagttttttcaacaaatagtgttgggacaatgggtgtccacatgcaaaagaataaagttgtccccttaccttacaccatctccaaaaattaactcaaaatatgtcaaagacataaacgtaagagctaaaactgtaaaactcctagaataaaacataggagtaaatcttcatgaccttggattaggccattgtgtcttaaata

SEQ ID NO: 26 - DNA, 370 bp, Homo Sapien
gaagccttactacaattccaaaaatcatcatggttggaaatttgggaggagattatttgtgaacttgttacccttttggtaatggtggactaattgctgtatagttatttttgttttattattactgttacattaatttaacatgcatttatagaagaatacattcaaagcactgatgtaggagatacacggtacttggagcagtcagccaaaaatcacagatactgctttcacttaaatggaaacaattctccgataatgctttgctttttttcttatgtcactcttgtgtactatctatttttctcctctctgggaccaagtttctttttataaagcaataatatctctgttttcatttcagaacatt

SEQ ID NO: 27 - DNA, 508 bp, Homo Sapien
gcttactatgtgctattgctgtgccaagtatctgaaacctgtcttcttaccttatttttcaagataattctatgtggcaggtattactatctcaattctaagagtgagaaaatggagttttagaaacatttactaacttgcctgggtcacatagctaaggaagaggtggacttgcccagctttgcataaaactcctcaaaagagttgcctatactccctgactccacttatcttcctactatcctctttttaaaatatattatttatttatttaaataagcaatatatgaatgtggtttgaaattcaaaagacacaaagaagtatacagaggaaagcctcactctcaatccttctcaaggtttgctaattcctcttgcataggcaatccgttcttccagctttgtgtttatctttccagagaagtttactgtgtattaagcaaatatgtatatctttattcttgctcagtattttcgcaaacagcagctgtctaagttcactgttctg

SEQ ID NO: 28 - DNA, 289 bp, Homo Sapien
gtactggcccttcggattgaaagtatacagtgatgaaatttgctgccactctttcatgcttggagtgttatattcttttggatgcgagccctcaaagaaacatttaatattctcttttgccaattcagttgcatgctctgtggctttacttttaaggatctgctgctcctgttccaaatagattttccagaatttcagctgcagaaaactaactggagataggcatcgggtgacagatgtaaaaatcagaagaatgatgataacaactgctatcaagatccagcccaac

SEQ ID NO: 29 - DNA, 299 bp, Homo Sapien
gtgttgggctggatcttgatagcagttgttatcatcattcttctgatttttacatctgtcacccgatgcctatctccagttagttttctgcagctgaaattctggaaaatctatttggaacaggagcagcagatccttaaaagtaaagccacagagcatgcaactgaattggcaaaagagaatattaaatgtttctttgagggctcgcatccaaaagaatataacactccaagcatgaaagagtggcagcaaatttcatcactgtatactttcaatccgaagggccagtactacagcat

SEQ ID NO: 30 - DNA, 428 bp, Homo Sapien
  misc_feature 75, 87, 115, 175, 258, 267, 310, 316, 328, 339, 352,366, 377, 398, 399: n = A,T,C or G
tagatttgcagttctgtggctcctgatggatctgagaagatggacgtggaggatgaaaatctgtctgattatttngaactgatgttngttgctatggagatgctgcctatatgtngatgttgcagacgttaagtcactagcccacagccttgtattccatactcagagaccctgntacttacttgacatctcaacttgaaagtccaattaatatgcacttcaaactttaataggcttcaaacagaatttctttcattnatctctgcnaaacagcttctctcatcatcttgaaattagtgaatggcatttnactgtnttagttggagtnatttctgtggntttctttcacatnctacataacaatcnatcagtaagtnctatgagctcttctttgaaannaaacagaatccaactgtttcattcccact

SEQ ID NO: 31 - DNA, 499 bp, Homo Sapien
  misc_feature 216, 303, 351: n = A,T,C or G
tcattttcctccacgtgaccaaatgagaaatttagtgaaagatttaaaatcatttttcagactttttccacattagttgggaagcaaaccccttttttaaggcaatgtcagttattaagctttagggaaccacatgccactttaggtaacacatgattggagagattgaagagtgaagtccctgctttaaagtgtactcctgtggacacagtaatncatatatttaaaatggttcatgttaagagtaggtatatttctatctaaatactctgtagcttttgtgattcagggaaatgagtggancctcacaggcacaagaatctagtaaattctaggtttcttgtgtggaantcagtgggcaaaatcttaactgagtgaattcttgattattggtatcacatttgttagtctgtatgtatctgtgtcatcgatctccttaagaagagactcgtagatattgactgggagacccaagctgaatgctaaaatctgctccatg

SEQ ID NO: 32 - DNA, 196 bp, Homo Sapien
  misc_feature 96, 97, 99, 100, 101, 102, 103, 104, 105, 106, 107, 108,109, 110, 111, 112, 113, 114, 115, 116, 117, 118, 120, 121,122, 123: n = A,T,C or G
cctctctagctgtgctttctatttttatgtgaggattcagggaaatccaaaaactatgctgctaccagcaccaccatcttctaggcatccagtganntnnnnnnnnnnnnnnnnnnnngnnnncatgtctttaactggtttgaatggacattgttggtttgagggaaaagatggggattaaacctcaagctaaaat

SEQ ID NO: 33 - DNA, 494 bp, Homo sapien
  misc_feature 101, 110, 367: n = A,T,C or G
tgaacttgttttatatttgcatatcaagagtcaagtatgatgtttttttaagttgactttttttacttcattatttttaggaataaatgtaagattttacnaatcttttnatttccccacaagatctgaagtttggtatttttgcattatgacagttgttgagactaggattttaagctaggatatgattatatttcctatataactaaaaattttgtttcataaattttaaaataattatttttgactatgaacattagtccaaatttaatatttgacacagttcataccagcttgctacaataatgataatttattagtctttctgttatttaaagaataaaaacatgcttataaaagacttttnaatgaaatgttgcctttttaaaataattatacttgcacatgaaaataaaatataaagtcaataatagtccttgtagcccaatgggaattgattctgtttattgtctgtaccattttgctaccagtta

SEQ ID NO: 34 - protein, 450 aa, Artificial Sequence
  Protein D-MAGE-A3 fusion protein
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 35 - DNA, 20 bp, Artificial Sequence
  CpG oligonucleotide sequence
tccatgacgttcctgacgtt

SEQ ID NO: 36 - DNA, 18 bp, Artificial Sequence
  CpG oligonucleotide sequence
tctcccagcgtgcgccat

SEQ ID NO: 37 - DNA, 30 bp, Artificial Sequence
  CpG oligonucleotide sequence
accgatgacgtcgccggtgacggcaccacg

SEQ ID NO: 38 - DNA, 24 bp, Artificial Sequence
  CpG oligonucleotide sequence
tcgtcgttttgtcgttttgtcgtt

SEQ ID NO: 39 - DNA, 20 bp, Artificial Sequence
  CpG oligonnucleotide sequence
tccatgacgttcctgatgct

SEQ ID NO: 40 - protein, 9 aa, Artificial Sequence
  MAGE peptide sequence
FLWGPRALV

SEQ ID NO: 41 - protein, 9 aa, Artificial Sequence
  MAGE peptide sequence
EVDPIGHLY

SEQ ID NO: 42 - protein, 10 aa, Artificial Sequence
  MAGE peptide sequence
MEVDPIGHLY

SEQ ID NO: 43 - protein, 10 aa, Artificial Sequence
  MAGE peptide sequence
VHFLLLKYRA

SEQ ID NO: 44 - protein, 10 aa, Artificial Sequence
  MAGE peptide sequence
LVHFLLLKYR

SEQ ID NO: 45 - protein, 10 aa, Artificial Sequence
  MAGE peptide sequence
LKYRAREPVT

SEQ ID NO: 46 - protein, 16 aa, Artificial Sequence
  MAGE peptide sequence
ACYEFLWGPRALVETS

SEQ ID NO: 47 - protein, 12 aa, Artificial Sequence
  MAGE peptide sequence
TQHFVQENYLEY

(19) United States
(12) Patent Application Publication
(10) Pub. No.: US 2010/0166783 A1
(43) Pub. Date: Jul. 1, 2010

(54) METHOD

(75) Inventors: Pierre Raphael Dupont, Louvain-la-Neuve (BE); Swann Romain Jean-Thomas Gaulis, Rixensart (BE); Thibault Marc Helleputte, Louvain-la-Neuve (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GlaxoSmithKline Biologicals SA, Rixensart (BE)

(21) Appl. No.: 12/558,943

(22) Filed: Sep. 14, 2009

Related U.S. Application Data
(60) Provisional application No. 61/192,042, filed on Sep. 15, 2008.

(30) Foreign Application Priority Data
Sep. 15, 2008 (GB) 0816867.6

(51) Int. Cl. A61K 39/00; C12Q 1/68; C40B 40/06

(52) U.S. Cl. 424/184.1; 435/6; 506/16

(57) ABSTRACT

Gene expression profiles, microarrays comprising nucleic acid sequences representing gene expression profiles, and new diagnostic kits and methods are provided. The gene expression profiles, microarrays, and new diagnostic kits and methods find use in the treatment of specific populations of cancer patients suffering from MAGE expressing tumours, the populations characterised by their gene expression profile.

### CROSS REFERENCE TO RELATED APPLICATIONS

This application claims the benefit of the co-pending international application filed 14 Sep. 2009, serial number to be provided, and claims the benefit of Great Britain patent application serial number 0816867.6, filed 15 Sep. 2008, and U.S. provisional application Ser. No. 61/192,042, filed 15 Sep. 2008, each of which are incorporated herein by reference in their entirety.

### FIELD OF THE INVENTION

The present invention relates to gene expression profiles; methods for classifying patients; microarrays; and treatment of populations of patients selected through use of methods and microarrays as described herein.

### INCORPORATION BY REFERENCE OF MATERIAL SUBMITTED ON COMPACT DISC

Applicants hereby incorporate-by-reference the material of the compact disc containing the files named: “How to import per q txt files into R session” created on 15 Sep. 2008 (file size 1 KB); “pe.RData” created on 15 Sep. 2008 (file size 22.801 MB); and “rq.RData” created on 15 Sep. 2008 (file size 5.254 MB). Applicants also hereby incorporate-by-reference the material of the compact disc containing the files named “pe.txt” created on 27 Oct. 2008 (file size 22,801,000 bytes); and “rq.txt” created on 27 Oct. 2008 (file size 5,254,000). A total of four compact discs (including duplicates) are incorporated by reference in the present paragraph, each filed in U.S. provisional application Ser. No. 61/192,042, filed 15 Sep. 2008.

### BACKGROUND

Melanomas are tumours originating from melanocyte cells in the epidermis. Patients with malignant melanoma in distant metastasis (stage 1V according to the American Joint Commission on Cancer (AJCC) classification) have a median survival time of one year, with a long-term survival rate of only 5%. Even the standard chemotherapy for stage 1V melanoma has therapeutic response rates of only 8-25%, but with no effect on overall survival. Patients with regional metastases (stage III) have a median survival of two to three years with very low chance of long-term survival, even after an adequate surgical control of the primary and regional metastases (Balch et al., 1992). Most Patients with stage I to III melanoma have their tumour removed surgically, but these patients maintain a substantial risk of relapse. Thus there remains a need to prevent melanoma progression, and to have improved treatment regimes for metastatic melanoma and adjuvant treatments for patients having had a primary tumour removed.

Traditional chemotherapy is based on administering toxic substances to the patient and relying, in part, on the aggressive uptake of the toxic agent by the tumour cells. These toxic substances adversely affect the patient's immune system, leaving the individual physically weakened and susceptible to infection.

It is known that not all patients with cancer respond to current cancer treatments. It is thought that only 30% or less of persons suffering from a cancer will respond to any given treatment. The cancers that do not respond to treatment are described as resistant. In many instances there have not been reliable methods for establishing if the patients will respond to treatment. However, administering treatment to patients who are non-responders because they cannot be differentiated from responders is an inefficient use of resources and, even worse, can be damaging to the patient because, as discussed already, many cancer treatments have significant side effects, such as severe immunosuppression, emesis and/or alopecia. It is thought that in a number of cases patients receive treatment, when it is not necessary or when it will not be effective.

Cells, including tumour cells, express many hundreds even thousands of genes. Differential expression of genes between patients who respond to a therapy compared to patients who do not respond, may enable specific tailoring of treatment to patients likely to respond.

### SUMMARY OF THE INVENTION

In one embodiment, the present invention provides a method for classifying a patient as a responder or non-responder to therapy, comprising measuring, in a patient-derived sample, the gene product of at least one gene selected from the genes listed in Table 1.

In a further embodiment, the present invention provides a method for classifying a patient as a responder or non-responder to therapy comprising measuring, in a patient-derived sample, the gene product recognised by a probe set selected from the probe sets listed in Table 1, the target sequences of which are shown in Table 4.

In a further embodiment, the present invention provides a method of characterising a patient as a responder or non-responder to therapy comprising the steps:

(a) analyzing a patient derived sample for differential expression of the gene products of one or more genes or immune response genes or a profile as described herein, and

(b) characterising the patient from which the sample was derived as a responder or non-responder to therapy, based on the results of step (a),

wherein the characterisation step is optionally performed by reference or comparison to a standard.

In a further embodiment, the present invention provides a microarray comprising one or more polynucleotide probes complementary and hybridisable to a sequence of the genes or immune mediated genes as described herein, in which polynucleotide probes or probe sets complementary and hybridisable to the genes or immune mediated genes constitute at least 50% of the probes or probe sets on said microarray.

In a further embodiment, the present invention provides a method of treating a patient characterised as a responder to therapy, comprising administering a therapy, vaccine or immunogenic composition as described herein to the patient.

In a further embodiment, the present invention provides a method of treating a patient characterised as a non-responder to a therapy according to methods described herein or use of a diagnostic kit as described herein, comprising administering an alternative therapy or a combination of therapies, for example chemotherapy and/or radiotherapy may be used instead of or in addition to a vaccine or immunogenic composition as described herein.

In a further embodiment, the present invention provides use of a composition comprising a tumour associated antigen in the preparation of a medicament for the treatment of patients characterised as responders according to methods described herein, use of a microarray as described herein, use of a gene profile as described herein or use of a diagnostic kit as described herein.

In one embodiment there is provided a gene profile which may be used to differentiate between a responder patient and a non-responder patient, wherein the profile comprises at least one gene selected from the genes listed in Table 1, or the profile comprises or consists of the genes listed in Table 1. In one aspect a gene profile according to the present invention comprises one or more genes of Table 1, or one or more genes recognised by the probe sets of Table 1. In a further aspect a profile comprises or consists of all the genes listed in Table 1 or comprises or consists of all the genes recognised or targeted by the probe sets listed in Table 1.

In one embodiment there is provided a gene profile as described herein, wherein the genes are genes recognised by the probe sets listed in Table 1.

In one aspect the invention provides a diagnostic kit comprising one or more nucleotide probes capable of hybridising to the gene products, for example mRNA or cDNA gene products, of the one or more of the genes listed in Table 1 or of the gene products of the genes listed in Table 1.

In one aspect the invention provides one or more probes for identifying gene products, for example mRNA or cDNA, of one or more genes of Table 1 or of the gene products of the genes listed in Table 1.

In one aspect the invention provides a microarray comprising one or more of the probes of Table 1 suitable for the detection of the gene products or gene profiles as described herein.

In another aspect the invention provides use of a microarray, including use of known microarrays, for the detection of gene products or gene profiles as described herein.

In one aspect the invention provides use of PCR (or other known techniques) for identification of differential expression (such as upregulation) of one or more of the gene products of Table 1, or of the gene products of the gene profiles as described herein.

### BRIEF DESCRIPTION OF THE FIGURES

FIG. 1/6: Sensitivity with random labels (with original labels in red—single vertical bar)—Bootstrap TP/P.

FIG. 2/6: Balance classification rate with random labels (with original labels in red—single vertical bar).

FIG. 3/6: Sensitivity with random labels (with original labels in red)—Bootstrap TN/N.

FIG. 4/6: Kaplan-Meier curve: Time to treatment failure by gene signature (GS− lower curve on figure; GS+ upper curve of figure).

FIG. 5/6: R code for GCRMA normalisation.

FIG. 6/6: Protein D 1/3-MAGE3-HIS protein.

### DETAILED DESCRIPTION

### Sequence Identifiers and Tables

The following sequence identifiers are included in the sequence listing:
SEQ ID NO:1-33—Probe set target sequences shown in Table 4
SEQ ID NO:34—Protein D-MAGE-A3 fusion protein
SEQ ID NO:35-39—CpG oligonucleotide sequences
SEQ ID NO:40-47—MAGE peptide sequences

As described in greater detail elsewhere, the following tables are set forth at the end of the description:
Table 1: 33 Probe set (PS) gene list
Table 2: Bootstrap 0.632+ performances of hard-margin linear SVMs built on the 33 PS reporter list of Table 1
Table 3: Indexes
Table 4: 33 PS target sequences
Table 5: 33 PS gene list showing up-regulated and down-regulated genes

### Predictive Gene Profile

Analysis performed on tumour tissue from patients having malignant melanoma, following surgical resection, identified that certain genes were differentially expressed in patients that were more likely to respond to therapy (responders), in comparison to those patients who were less likely to respond (non-responders).

The present inventors have discovered a gene profile that is predictive of the likelihood of a patient's response to therapy.

By “gene profile” is intended a gene or a set of genes the expression of which correlates with patient response to therapy because the gene or set of genes exhibit(s) differential expression between patients having a favourable response to therapy and patients having a poor response to therapy. In one embodiment of the invention the term “gene profile” refers to the genes listed in Table 1 or to any selection of the genes of Table 1 which is described herein.

“Differential expression” in the context of the present invention means the gene is up-regulated or down-regulated in comparison to its normal expression. Statistical methods for calculating differential expression of genes are discussed elsewhere herein.

The invention provides a gene profile for characterising a patient as a responder or non-responder to therapy, in which the profile comprises differential expression of at least one gene of Table 1, or in which the profile comprises or consists of the genes listed in Table 1. A profile may be indicative of a responder or non-responder. In one embodiment, the gene profiles described herein are indicative of responders.

The gene sequences recognised or targeted by the probe sets of Table 1 are listed in Table 4.

A gene profile may comprise differential expression of at least 5, at least 10, at least 15, at least 20 and/or all the genes of Table 1. In one embodiment, at least 80% of the gene products of the gene profile are upregulated. In one embodiment, 82% of the gene products are upregulated and 18% are down-regulated.

In one embodiment there is provided a gene profile as described herein, in which at least one gene(s) is upregulated. In one embodiment there is provided a gene profile as described herein, in which gene products of 27 genes are upregulated and gene products of 6 genes are down-regulated.

In one embodiment of the present invention, there is provided a gene profile comprising the genes of Table 1 or the genes recognised by the probe sets of Table 1, in which the gene products of the target genes of the gene profile are upregulated or downregulated as shown in Table 5.

For the predictive gene profile identified herein, the microenvironment of the tumour may be key to whether the patient has a good or poor prognosis, following surgical resection, or may be key to whether the patient is a responder to therapy.

In one embodiment of the invention, the genes to be analysed for differential expression are the immune-related genes shown in Table 1. In one embodiment, the one or more genes of Table 1 are the immune related genes shown in Table 1.

By “genes of Table 1” is meant the gene products of genes listed under “Gene name” in Table 1. By “gene product” is meant any product of transcription or translation of the genes, whether produced by natural or artificial means.

In one embodiment of the invention, the genes referred to herein are those listed in Table 1 as defined in the column 2, “Gene name”. In another embodiment, the genes referred to herein are genes the product of which are capable of being recognised by the probe sets listed in column 1 of Table 1.

“Therapy” in the context of the invention can mean treatment with a composition comprising a tumour-associated antigen. Alternatively, therapy may comprise treatment with an antibody that specifically binds to the tumour associated antigen. Such treatment may result in the treatment, amelioration and/or retardation of the progression of a disease associated therewith. Treatment in this context may include adjuvant-stage treatment, in which the therapy is given after surgical resection of the tumour tissue. Antigens that may be used in such therapy include those described herein.

This invention may be used for identifying cancer patients that are likely to respond to therapy, for example patients with melanoma, breast, bladder, lung, NSCLC, head and neck cancer, squamous cell carcinoma, colon carcinoma and oesophageal carcinoma, such as in patients with MAGE-expressing cancers. In an embodiment, the invention may be used in an adjuvant (post-operative, for example substantially disease-free) setting in such cancers, particularly lung and melanoma. The invention also finds utility in the treatment of cancers in the metastatic setting.

Thus in one aspect the invention provides a predictive gene profile indicative of a patient's likelihood of response to a therapy, comprising differential expression of one or more of the gene products of Table 1 or of a gene profile described herein.

In a further aspect the invention provides a gene profile indicative of a patient's likelihood of response to a therapy, in which the patient expressing the profile may be characterised as being a responder or non-responder, the gene profile comprising differential expression of one or more of the genes listed in Table 1, or the gene profile comprising or consisting of the genes listed in Table 1.

In one embodiment, a gene profile as described herein may be a predictive gene profile, which is indicative of a likelihood of response to therapy.

“Immune activation gene” is intended to mean a gene whose product (eg mRNA or protein expressed from the gene) facilitates, increases, stimulates or is associated with the immune response. “Immune response gene”, “immune activation gene” and “immune related genes” are used interchangeably herein.

### Measurement of a Gene Profile/Level of Differential Expression

In the context of the present invention, the term “gene product” is intended to mean the mRNA or protein encoded by a gene, or cDNA that corresponds to the encoded mRNA.

An important technique for the analysis of the genes expressed by cells, such as cancer/tumour cells, is DNA microarray (also known as gene chip technology), where hundreds or more probe sequences (such as 55,000 probe sets) are attached to a glass surface. The probe sequences are generally all 25 mers or 60 mers and are sequences from known genes. These probes are generally arranged in a set of 11 individual probes (a probe set) and are fixed in a predefined pattern on the glass surface. Once exposed to an appropriate biological sample these probes hybridise to the relevant RNA or DNA of a particular gene. After washing, the chip is “read” by an appropriate method and a quantity such as colour intensity recorded. The differential expression of a particular gene is proportional to the measure/intensity recorded. This technology is discussed in more detail below.

Another useful technique for the measurement of protein gene products is through use of proteomic technology.

Once a target gene/profile has been identified there are several analytical methods to measure whether the gene(s)/profile(s) is/are differentially expressed. For DNA, these analytical techniques include real-time polymerase chain reaction, also called quantitative real time polymerase chain reaction (QRT-PCR or Q-PCR), which is used to simultaneously quantify and amplify a specific part of a given DNA molecule present in the sample.

The procedure follows the general pattern of polymerase chain reaction, but the DNA is quantified after each round of amplification (the “real-time” aspect). Two common methods of quantification are the use of fluorescent dyes that intercalate with double-strand DNA, and modified DNA oligonucleotide probes that fluoresce when hybridized with a complementary DNA.

The basic idea behind real-time polymerase chain reaction is that the more abundant a particular cDNA (and thus mRNA) is in a sample, the earlier it will be detected during repeated cycles of amplification. Various systems exist which allow the amplification of DNA to be followed and they often involve the use of a fluorescent dye which is incorporated into newly synthesised DNA molecules during real-time amplification. Real-time polymerase chain reaction machines, which control the thermocycling process, can then detect the abundance of fluorescent DNA and thus the amplification progress of a given sample. Typically, amplification of a given cDNA over time follows a curve, with an initial flat-phase, followed by an exponential phase. Finally, as the experiment reagents are used up, DNA synthesis slows and the exponential curve flattens into a plateau.

Alternatively the mRNA or protein product of the target gene(s) may be measured by Northern Blot analysis, Western Blot and/or immunohistochemistry.

In one aspect the methods or analyses described herein are performed on tumour samples in which a tumour associated antigen, for example a cancer testis antigen, is further expressed.

When a single gene is analysed by, for example, Q-PCR then the gene expression can be normalised by reference to a gene that remains constant, for example genes with the symbol H3F3A, GAPDH, TFRC, GUSB or PGK1. The normalisation can be performed by subtracting the value obtained for the constant gene from the value obtained for the gene under consideration.

A threshold may be established by plotting a measure of the expression of the relevant gene for each patient. Generally the responders and non-responders will be clustered about different axes/focal points. A threshold can be established in the gap between the clusters by classical statistical methods or simply plotting a “best fit line” to establish the middle ground between the two groups. Values, for example, above the pre-defined threshold can be designated as responders and values, for example below the pre-designated threshold can be designated as non-responders.

In one embodiment, the genes of a gene profile described herein are differentially expressed.

Once at least two differentially expressed genes are included in a profile then statistical clustering methods can be used to differentiate the patients likely to respond to therapy “responders” from patients unlikely or less-likely to respond to therapy “non-responders”.

In one aspect the invention provides a gene profile for identifying a responder comprising one or more of the genes of Table 1. In one embodiment, the invention provides a gene profile comprising at least 5, at least 10, at least 15, at least 20, at least 30 and/or all the genes of Table 1. In a further embodiment, the invention provides a gene profile comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the genes of Table 1.

In one embodiment of the methods described herein, the genes listed in Table 1 are up-regulated and down-regulated respectively, as indicated in Table 5, in patients designated as responders.

### Statistical Methods

Methods for statistical clustering and software for the same are discussed below.

One parameter used in quantifying the differential expression of genes is the fold change, which is a metric for comparing a gene's mRNA-expression level between two distinct experimental conditions. Its arithmetic definition differs between investigators.

However, the greater the fold change the more likely that the differential expression of the relevant genes will be adequately separated, rendering it easier to decide which category a patient falls into.

The fold change for an upregulated gene may be, for example, at least 1.4, at least 1.5, at least 1.6, at least 1.7, at least 1.8, at least 1.9 or at least 2.0. In one embodiment, in which the expression level is measured using PCR, the fold change is at least 2.0.

The fold change for a down-regulated gene may be 0.6 or less than 0.6, for example it may be 0.5 or less than 0.5, 0.4 or less than 0.4, 0.3 or less than 0.3, 0.2 or less than 0.2 or may be 0.1 or less than 0.1.

A fold change of 0.1 indicates that the expression of a gene is down-regulated 10 times. A fold change of 2.0 indicates that the expression of a gene is upregulated 2 times.

In the present examples, fold change is calculated as the signal in responder patients/signal in non-responder patients. Therefore, the fold changes shown in the tables are indicative of a likelihood of response to therapy.

For example:
If the fold change of a gene=2, the signal in the group of responder patients (responders) is 2× greater than in the group of non-responder patients (non-responders). The up-regulation of the gene is linked to a favourable clinical response.
If the fold change of a gene=0.5, the signal in the group of responder patients (responders) is 2× smaller than in the group of non-responder patients (non-responders). The down-regulation of the gene is linked to a favourable clinical response.

Another parameter also used to quantify differential expression is the “p” value. It is thought that the lower the p value the more differentially expressed the gene is likely to be, which renders it a good candidate for use in profiles of the invention. P values may for example include 0.1 or less, such as 0.05 or less, in particular 0.01 or less. P values as used herein include corrected “P” values and/or also uncorrected “P” values.

As used herein, a ‘favourable response’ (or ‘favourable clinical response’) to, for example, an anticancer treatment refers to a biological or physical response that is recognized by those skilled in the art as indicating a decreased rate of tumour growth, compared to tumour growth that would occur with an alternate treatment or the absence of any treatment. “Favourable clinical response” as used herein is not synonymous with a cure, but includes Partial Response, Mixed Response or Stable Disease. A favourable clinical response to therapy may include a lessening of symptoms experienced by the subject, an increase in the expected or achieved survival time, a decreased rate of tumour growth, cessation of tumour growth (stable disease), regression in the number or mass of metastatic lesions, and/or regression of the overall tumour mass (each as compared to that which would occur in the absence of therapy, or in response to an alternate therapy).

“Non-responder” in the context of this invention includes persons whose symptoms ie cancers/tumours are not improved or stabilised.

“Responder” in the context of the present invention includes persons where the cancer/tumour(s) is eradicated, reduced or improved (mixed responder or partial responder) by therapy, or simply stabilised such that the disease is not progressing. In responders where the cancer is stabilised then the period of stabilisation is such that the quality of life and/or patients life expectancy is increased (for example stable disease for more than 6 months) in comparison to a patient that does not receive treatment.

Partial clinical response in respect of cancer is wherein all of the tumours/cancers respond to treatment to some extent, for example where said cancer is reduced by 30, 40, 50, 60% or more.

Mixed clinical responder in respect of cancer is defined as wherein some of the tumours/cancers respond to treatment and others remain unchanged or progress.

Standard definitions are available for responders, partial responders and mixed responders to cancer treatment. These standard definitions apply herein unless from the context it is clear that they do not apply.

“Training set” in the context of the present specification is intended to refer to a group of samples for which the clinical results can be correlated with the a profile and can be employed for training an appropriate statistical model/programme to assign favourable clinical response/responder or non-responder for new samples.

Table 3 contains values of medians, iqrs and w for the 33 probe set model of Table 1, as described in the Examples.

### Predictive Methods

The invention provides a method for predicting the likelihood of response of a patient to a therapy, the method comprising analysis of the expression of gene products of a gene profile as described herein.

The invention provides a method for the detection of a gene profile in a biological sample, the method comprising measuring the expression of at least 5, at least 10, at least 15, at least 20, at least 30 and/or all the gene products recognised by the probe sets listed in Table 1. Alternatively, the method may comprise measuring the expression of the gene product recognised by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the probe sets listed in Table 1 and/or any combination thereof.

In one embodiment of the present invention there is provided a method for classifying a patient as a responder or non-responder to therapy comprising measuring, in a patient-derived sample, the gene product of at least one gene selected from the genes listed in Table 1. The method may comprise measuring the gene product of at least 5, at least 10, at least 15, at least 20, at least 30 and/or all the genes recognised by the probe sets listed in Table 1 and/or any combination thereof. Alternatively, the method may comprise measuring the gene product of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the genes of Table 1 or the genes recognised by the probe sets listed in Table 1.

The methods described herein may further comprise the step of determining whether the gene products are upregulated and/or downregulated as described herein, or as shown in Table 5.

Thus in one aspect the invention provides a method of identifying whether a patient is a responder or non-responder to therapy, following surgical resection of a tumour, the method comprising: analysing a sample for differential expression of one or more genes or gene profiles as described herein; and characterising a patient as being a responder or non-responder.

Thus in one aspect the invention provides a method of identifying whether a patient will be a responder or non-responder to therapy, the method comprising: (a) analysing a patient-derived sample comprising mRNA or fragments thereof expressed by genes of cancerous cells or DNA or fragments thereof from cancerous cells, for differential expression of one or more genes selected from the group comprising or consisting of genes listed in Table 1 or for differential expression of the genes comprising or consisting of the genes listed in Table 1; and (b) characterising a patient as a responder or a non-responder based on the results of step (a).

In one embodiment of the invention described herein, the patient is a patient suffering from cancer or having a tumour, or a patient having had surgical removal or resection of a tumour or tumour tissue.

As described herein, methods to predict a favourable clinical response or to identify subjects more likely to respond to therapy, is not meant to imply a 100% predictive ability, but to indicate that subjects with certain characteristics are more likely to experience a favourable clinical response to a therapy than subjects who lack such characteristics. However, as will be apparent to one skilled in the art, some individuals identified as more likely to experience a favourable clinical response may nonetheless fail to demonstrate measurable clinical response to the treatment. Similarly, some individuals predicted as non-responders may nonetheless exhibit a favourable clinical response to the treatment.

Optionally the characterisation of the patient as a responder or non-responder can be performed by reference to a “standard” or a training set. The standard may be a profile of a person/patient who is known to be a responder or non-responder or alternatively may be a numerical value. Such pre-determined standards may be provided in any suitable form, such as a printed list or diagram, computer software program, or other media.

Patients whose tumour tissue expresses a tumour associated antigen as described herein, who additionally exhibit a gene profile described herein as a responder profile, may be selected for treatment with a composition comprising a tumour associated antigen.

For example, patients whose tumour tissue is found or is known to express a MAGE antigen, and who exhibit a gene profile described herein as a responder profile, may be selected for therapy with a composition comprising a MAGE antigen.

In one aspect a mathematical model/algorithm/statistical method is employed to characterise the patient as responder or non-responder.

In one embodiment there is provided a method for classifying a patient as a responder or non-responder comprising the steps of:

(a) determining in a patient-derived sample the level of expression of a gene product of at least one gene selected from the genes listed in Table 1; and

(b) classifying the patient as a responder or non-responder from the expression levels determined under (a).

In one embodiment, the method comprises determining the level of expression of the gene product of at least 5, at least 10, at least 15, at least 20, at least 30 and/or all the genes recognised by the probe sets listed in Table 1 and/or any combination thereof. In one embodiment, the method comprises determining the level of expression of the gene product of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the genes recognised by the probe sets listed in Table 1.

In one embodiment, step (b) is based on a mathematical discriminant function or a decision tree. The decision tree may involve at least one bivariate classification step. In another embodiment, a k-nearest-neighbour (kNN) algorithm may be used in step (b). Alternatively, classification can be achieved using other mathematical methods that are well known in the art.

In one embodiment, step (b) comprises use of a k-nearest-neighbour (kNN) algorithm.

In one aspect the invention provides a profile based on the genes of Table 1. In one aspect the invention provides a profile based on the 33 probe sets listed in Table 1.

In one embodiment the gene profile as described herein is predictive of a patient's likelihood to respond to therapy following surgical resection of a tumour.

In one embodiment there is provided a gene profile, in which one or more, for example at least 5, for example at least 10, at least 15, at least 20, at least 30 and/or all of the genes of Table 1 or genes recognised by the probe sets listed in Table 1 are differentially expressed.

The invention herein extends to use of all permutations of any and all of the genes listed herein for identification of a profile/profile as described herein.

The invention additionally extends to functional equivalents of genes listed herein, for example as characterised by hierarchical classification of genes such as described by Hongwei Wu et al 2007 (Hierarchical classification of equivalent genes in prokaryotes—Nucleic Acid Research Advance Access).

The genes described herein were identified by specific probes and a skilled person will understand that the description of the genes above is a description based on current understanding of what hybridises to the probe. However, regardless of the nomenclature used for the genes by repeating the hybridisation to the relevant probe under the prescribed conditions the requisite gene may be identified. Whilst not wishing to be bound by theory, it is thought that is not necessarily the gene per se that is characteristic of a profile but rather it is the gene function which is fundamentally important. Thus a functionally equivalent gene to a gene listed herein may be employed in a profile.

The invention extends to use of profile(s) according to the invention for predicting or identifying a patient as a responder or non-responder to therapy.

Thus the invention includes a method of analyzing a patient derived sample, based on differential expression of a gene profile according to the invention for the purpose of characterising the patient from which the sample was derived as a responder or non-responder to therapy.

In one aspect the invention provides a method of characterising a patient as a responder or non-responder comprising the steps:

a) analysing a patient derived sample for differential expression of one or more gene products or a gene profile as described herein, and

b) characterising the patient from which the sample was derived as a responder or non-responder to therapy, based on the results of step (a).

In one embodiment, the characterisation step may be performed by reference or comparison to a standard.

Suitable standards are described herein although other suitable standards are known and may be contemplated by those skilled in the art.

In one aspect the method of the present invention additionally provides the step of selecting patients identified as responders by methods described herein for therapy.

In one aspect the present invention relates to a method for the detection of a gene profile in a biological sample, the method comprising the analysis of the expression of the gene products of one or more genes of Table 1. Alternatively, the method may comprise the analysis of the expression of the gene products of one or more genes recognised by the probe sets of Table 1.

The present invention therefore, generally relates, in one aspect, to a method for the detection of a gene profile in a biological sample, the method comprising the analysis of the expression of the genes of Table 1 or of the genes recognised by the probe sets of Table 1, or of the genes of a gene profile described herein.

In one aspect the invention provides a method for measuring expression levels of polynucleotides from genes such as one or more genes listed in Table 1 or genes recognised by probe sets listed in Table 1 or a gene profile as described herein in a patient-derived sample for the purpose of identifying if the patient is likely to be a responder or non-responder to therapy comprising the steps: isolating RNA from the sample; preparing cDNA corresponding to the isolated RNA and optionally amplifying the cDNA; and quantifying the levels of cDNA in the sample.

In one aspect the invention provides a method for measuring expression levels of polynucleotides from the immune related genes listed in Table 1 in a patient-derived sample for the purpose of identifying if the patient is likely to be a responder or non-responder to therapy comprising the steps: isolating RNA from the sample; preparing cDNA corresponding to the isolated RNA and optionally amplifying the cDNA; and quantifying the levels of cDNA in the sample.

In one embodiment, the diagnostic method comprises determining whether a patient-derived sample expresses any of the gene products of the genes set forth in Table 1 or any other embodiment of the invention described herein by, for example, detecting the level of the corresponding RNA, for example mRNA, or corresponding cDNA, and/or protein level of the gene products of the genes set forth in Table 1 or genes recognised by the probe sets of Table 1.

The diagnostic method as described herein may be performed using microarray technology, for example gene chip technology, or by techniques such as by Northern blot analysis, reverse transcription-polymerase chain reaction (RT-PCR), in situ hybridization, immunoprecipitation, Western blot hybridization, or immunohistochemistry.

According to the methods described herein, tumour tissue may be obtained from a subject (a “patient-derived sample”) and levels of protein, mRNA or corresponding cDNA (gene product) of the genes may be analysed according to methods described herein.

Patients whose tissue is found to differentially express one or more or all of the genes of Table 1 or genes described in another embodiment of the invention may be predicted to respond or not respond to therapy. Patients determined to be responders may benefit from therapies described herein, for example treatment with a composition comprising a tumour associated antigen. Patients determined not to be responders may benefit from alternative treatment approaches.

A nucleic acid molecule which hybridises to a given location on a microarray may be “differentially expressed” and indicative of a responder, according to the present invention if the hybridisation signal is, for example, higher than the hybridisation signal at the same location on an identical array hybridised with a nucleic acid sample obtained from a subject that does not respond to therapy (a non-responder).

Kits for use in the methods of the present invention are also provided by the present invention. These may comprise materials/reagents for PCR (such as QPCR), microarray analysis, immunohistochemistry or other analytical techniques that may be used for determining differential expression of one or more of the genes of Table 1.

The invention also provides a diagnostic kit comprising a set of probes capable of hybridising to the mRNA or cDNA of one or more or all of the genes of Table 1 or one or more or all of the target sequences of the probe sets of Table 1. For example, the kit may comprise a set of probes capable of hybridising to the mRNA or its cDNA of one or more or all of the genes of Table 1 or of one or more or all of the target sequences of the probe sets of Table 1 or any other embodiment of the invention as described herein.

In another embodiment the present invention provides a diagnostic kit comprising a microarray substrate and probes that are capable of hybridising to the mRNA or cDNA of one or more or all of the genes of Table 1 or one or more or all of the target sequences of the probe sets of Table 1 or any other profile of the invention as described herein.

In one aspect the invention provides microarrays adapted for identification of a profile according to the invention. The invention also extends to substrates and probes suitable for hybridising to an mRNA or cDNA moiety expressed from one or more genes described herein, or genes having target sequences recognised by probe sets described herein.

Commercially available microarrays contain many more probes than are required to characterise the differential expression of the genes under consideration at any one time, to aid the accuracy of the analysis. Thus one or more probe sets may recognise the same gene. Thus in one embodiment multiple probes or probe sets may be used to identify if a gene described herein is differentially expressed, for example upregulated, as described herein.

The diagnostic kit may, for example comprise probes, which are arrayed in a microarray.

Specifically, prepared microarrays, for example, containing one or more probe sets described herein can readily be prepared by companies such as Affymetrix, thereby providing a specific test and optionally reagents for identifying a profile, according to the invention. In one aspect, this invention relates to oligonucleotide probes and primers capable of recognising the gene products of the genes of Table 1 or any other gene profile as described herein and diagnostic kits based on these probes and primers.

In an embodiment the methods, microarrays or diagnostic kits described herein are additionally able to test for the presence or absence of tumour associated antigen gene product, for example the gene product of one or more of the following antigens: a MAGE antigen, in which the MAGE antigen may be selected from MAGE A1, MAGE A2, MAGE A3, MAGE A4, MAGE A5, MAGE A6, MAGE A7, MAGE A8, MAGE A9, MAGE A10, MAGE A11, MAGE A12, MAGE-B1, MAGE-B2, MAGE-B3 and MAGE-B4, MAGE-C1 and MAGE-C2. The MAGE A antigens listed above are also known by the following nomenclature: MAGE 1, MAGE 2, MAGE 3, MAGE 4, MAGE 5, MAGE 6, MAGE 7, MAGE 8, MAGE 9, MAGE 10, MAGE 11, MAGE 12, and both terms are used interchangeably herein; MAGE-B1, MAGE-B2, MAGE-B3 and MAGE-B4, MAGE-C1 and MAGE-C2; Her-2/neu; P501S; WT-1; PRAME; LAGE 1; NY-ESO-1; SSX-2; SSX-4; SSX-5; NA17; MELAN-A; Tyrosinase; P790; P510; P835; B305D; B854; CASB618; CASB7439 (HASH-2); C1491; C1584; and C1585.

The invention herein described extends to use of all permutations of the probes listed herein (or functional analogues thereof) for identification of a profile as described herein.

In one aspect the invention provides use of a probe for the identification of differential expression of at least one gene product of an immune activation gene for establishing if a gene profile according to the present invention is present in a patient-derived sample.

Hybridisation may be performed under stringent conditions, such as 3× saline/sodium citrate (SSC), 0.1% SDS, at 50° C. In one embodiment, hybridization conditions for cDNA microarrays are hybridisation in 5×SSC or 6×SSPE with 0.1-0.2% SDS at 37° C.-65° C. (depending on the manufacturer recommended conditions for the probes) for four hours, followed by washes at 25° C. in low stringency wash buffer (1×SSC, 0.2% SDS), followed by 10 minutes at 25° C. in higher stringency wash buffer (0.1×SSC, 0.2% SDS).

Once target gene(s)/profile(s) has/have been identified then it is well within the skilled person's ability to design alternative probes that hybridise to the same target. Therefore the invention also extends to probes which under appropriate conditions measure differential expression of the gene(s) of the present invention to provide a profile/profile as described.

The invention also extends to use of the relevant probe in analysis of whether a patient is a responder or non-responder, for example through use of methods as described herein.

The invention also extends to the use of known microarrays for identification of a profile as described herein and methods using such profiles.

A nucleic acid probe may be at least 10, 15, 20, 25, 30, 35, 40, 50, 75, 100 or more nucleotides in length and may comprise the full length gene. Probes for use in the invention are those that are able to hybridise specifically to the mRNA or cDNA of the genes of Table 1 under stringent conditions.

The present invention further relates to a method of screening the effects of a drug or therapy on a patient's predicted response, comprising the step of analysing a profile as described herein before and after administration of a drug or therapy to a patient. The invention therefore provides a method for screening for a drug or therapy which alters a profile to that of a patient having favourable predicted response. The drug or therapy may be administered 1, 2, 3, 4, 5, 6, 7, 8, 9, 10, 11, 12, 13 or 14 days before a patient-derived sample is subject to a screening or diagnostic method as described herein.

In one embodiment, administration of a drug or therapy may be used to alter a profile to that of a patient predicted to respond to therapy, as described herein. The drug or therapy may comprise the drug or therapy identified through the method of screening described above. In one embodiment, the drug or therapy comprises topical administration of imiquimod: such topical administration is particularly suitable for a gene profile of external lesions or tumours, for example skin lesions. In one embodiment, the drug or therapy is local irradiation of the tumour. In one embodiment, the drug or therapy is selected from the group comprising: IL-2, IFN-α, dimethyltrizenoimidazolcarboxam (dacarbazine; DTIC) and temozolomide (TMZ).

In one embodiment, the table below describes possible drug or therapy administration that may be used to alter a profile:

| Tumour | | |
|---|---|---|
| type | Tumour stage | Treatment |
| Metastatic | Unresectable | DTIC or TMZ as first-line treatment |
| melanoma | stage III or IV | First-line chemotherapy treatment other |
| | | than DTIC only or TMZ only (that may, |
| | | but need not, include DTIC, TMZ, IL-2 |
| | | or IFNα) |
| | | Any second-line chemotherapy |
| | | treatment |
| | | Local irradiation of cutaneous/ |
| | | subcutaneous tumour lesions |
| | | Local imiquimod |
| Non-small- | Any stage, if the | Chemo(radio)-therapy induction doublet |
| cell lung | patient is eligible | neo-adjuvant chemotherapy with |
| cancer | for neo-adjuvant | platinum plus a second chemotherapy |
| | chemotherapy | drug. |
| | with subsequent | [Note: Thus, induction radiotherapy is |
| | resection | permitted.] |

DTIC, dimethyltrizenoimidazolcarboxam (dacarbazine);
TMZ, temozolomide.

The present invention further provides a method of patient diagnosis comprising the step of analysing whether a patient-derived sample expresses a gene profile as described herein and comparing it with a pre-determined standard to determine whether the patient is a responder and would benefit from additional therapy, for example administration of a composition comprising a tumour associated antigen. In one embodiment, the “standard” could be a patient-derived sample or samples from patients known to respond or not respond to a therapy.

In a further embodiment, the term “expresses a gene profile” or “differential expression of a gene profile” as used herein is used to described a patient-derived sample in which the genes of a gene profile are differentially expressed in a patient who responds to or is predicted to respond to therapy in comparison to a patient who does not respond to or is predicted not to respond to therapy, or in comparison to a pre-determined standard, as required.

The invention includes a method of predicting a patient's response to a therapy comprising the step of analysing a patient-derived sample for differential expression of a gene profile as described herein.

The invention includes a method of patient diagnosis comprising the step of analysing a patient-derived sample for differential expression of a gene profile as described herein.

The invention includes a method of patient diagnosis comprising the step of analysing the expression profile of at least 5, at least 10, at least 15, at least 20, at least 30 and/or all the genes of Table 1 or other embodiment of the invention from a patient-derived sample and assessing whether the genes are expressed and/or differentially expressed.

### Samples

Thus in clinical applications, tissue samples from a human patient may be screened for the presence and/or absence of differential expression of a gene profile as described herein.

In the context of the present invention, the sample may be of any biological tissue or fluid derived from a patient potentially in need of treatment. The sample maybe derived from sputum, blood, urine, or from solid tissues such as biopsy from a primary tumour or metastasis, or from sections of previously removed tissues.

Samples could comprise or consist of, for example, needle biopsy cores, surgical resection samples or lymph node tissue. These methods include obtaining a biopsy, which is optionally fractionated by cryostat sectioning to enrich tumour cells to about 80% of the total cell population. In certain embodiments, nucleic acids extracted from these samples may be amplified using techniques well known in the art. The levels of selected markers in a profile can be detected and can be compared with statistically valid groups of, for example, MAGE positive non responder patients.

For cancer, the biological sample may contain cancer or tumour cells and may, for example, be derived from the cancer or tumour such as a fresh sample (including frozen samples) or a sample that has been preserved in paraffin. Having said this, samples preserved in paraffin can suffer from degradation and a profile observed may be modified. A person working the in field is well able to compensate of these changes observed by recalibrating the parameters of the profile.

### Microarrays

A microarray is an array of discrete regions, typically nucleic acids, which are separate from one another and are typically arrayed at a density of between, about 100/cm² to 1000/cm², but can be arrayed at greater densities such as 10000/cm². The principle of a microarray experiment, is that mRNA from a given cell line or tissue is used to generate a labeled sample typically labeled cDNA, termed the ‘target’, which is hybridized in parallel to a large number of, nucleic acid sequences, typically DNA sequences, immobilised on a solid surface in an ordered array.

Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25 mers are synthesized in situ, either by photolithography onto silicon wafers (high-density-oligonucleotide arrays from Affymetrix or by ink-jet technology (developed by Rosetta Inpharmatics, and licensed to Agilent Technologies).

Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100 mers) has recently been developed to counteract these disadvantages.

Thus in performing a microarray to ascertain whether a patient-derived sample expresses a gene profile of the present invention, the following steps are performed: obtain mRNA from the sample and prepare nucleic acids targets, contact the array under conditions, typically as suggested by the manufactures of the microarray (suitably stringent hybridisation conditions such as 3×SSC, 0.1% SDS, at 50° C.) to bind corresponding probes on the array, wash if necessary to remove unbound nucleic acid targets and analyse the results.

It will be appreciated that the mRNA may be enriched for sequences of interest such as those present in a gene profile as described herein by methods known in the art, such as primer specific cDNA synthesis. The population may be further amplified, for example, by using PCR technology. The targets or probes are labeled to permit detection of the hybridisation of the target molecule to the microarray. Suitable labels include isotopic or fluorescent labels which can be incorporated into the probe.

In an alternative embodiment, a patient may be diagnosed to ascertain whether his/her tumour expresses a gene profile of the invention using a diagnostic kit based on PCR technology, in particular Quantative PCR (For a review see Ginzinger D Experimental haematology 30 (2002) p 503-512 and Giuliette et al Methods, 25 p 386 (2001).

In an alternative aspect the invention provides a method described herein, further comprising the steps of analysing a tumour derived sample to determine which antigen(s) are expressed by the tumour, and administrating a composition comprising the antigen(s). For example where the tumour is found to express a MAGE antigen, the composition may comprise a MAGE antigen.

In a further aspect the invention provides a method of treating a patient suffering from a tumour, in which the patient is designated as a responder through any method described herein, the method comprising treatment with an appropriate therapy, for example administration of a composition comprising a tumour associated antigen.

Such treatment or therapy may be given after resection by surgery of any tumour or after chemotherapy or radiotherapy treatment.

A further aspect of the invention is a method of treating a patient suffering from a tumour, for example a MAGE antigen expressing tumour, the method comprising determining whether a patient's tumour expresses a gene profile as described herein and then administering a composition comprising a tumour associated antigen.

Also provided is a method of treating a patient susceptible to recurrence of a tumour, for example a MAGE antigen expressing tumour, in which the patient has had tumour tissue resected, the method comprising: determining whether a patient is a responder according to methods described herein and then administering a composition comprising a tumour associated antigen.

Also provided is a method of treating a patient susceptible to recurrence of MAGE expressing tumour, the patient having had tumour tissue resected, the method comprising determining whether the patient's resected tumour tissue expresses at least 5, at least 10, at least 15, at least 20, at least 30 and/or all genes selected from Table 1, and then administering a composition comprising a tumour associated antigen.

The invention further provides the use of composition as described herein in the manufacture of a medicament for the treatment of patients determined to be a responder according to methods described herein.

In one aspect the invention provides use of a composition as described herein in the manufacture of a medicament for the treatment of patients suffering from or susceptible to recurrence of a tumour, in which the patient's tumour tissue expresses a gene profile or gene product(s) as described herein.

### Therapy

The term “therapy” as used herein may refer to administration of, or treatment with, a composition comprising a tumour associated antigen, as described herein. The therapy as described herein may be used or administered to prevent or ameliorate recurrence of disease. Such treatment may be given after resection by surgery of any tumour or after chemotherapy or radiotherapy treatment. Therapy may also include treatment of a patient with a combination of therapies, for example chemotherapy and/or radiotherapy may be used instead of or in addition to the composition comprising the tumour associated antigen as described herein

As used herein, the term “tumour associated antigen” means an antigen that is highly correlated with tumour cells. For example, the antigen may be over-expressed in tumour cells, in comparison with normal cells; the antigen may be tissue-specific, for example limited in expression to prostate cells such that after removal of the prostate the only cells will be tumour cells derived from prostate tissue; or the antigens may be tumour-specific antigens, such that the only cells on which the antigens are expressed are tumour cells.

In one embodiment, the tumour associated antigen may be an antigen or a derivative thereof. For example, the antigen may be a MAGE antigen or derivative as described herein, in which the MAGE antigen may be selected from MAGE A1, MAGE A2, MAGE A3, MAGE A4, MAGE A5, MAGE A6, MAGE A7, MAGE A8, MAGE A9, MAGE A10, MAGE A11, MAGE A12, MAGE-B1, MAGE-B2, MAGE-B3 and MAGE-B4, MAGE-C1 and MAGE-C2. Alternatively, the tumour associated antigen may be selected from: Her-2/neu; P501S; WT-1, for example the fragment WT-1F; PRAME; LAGE 1; NY-ESO-1; SSX-2; SSX-4; SSX-5; NA17; MELAN-A; Tyrosinase; P790; P510; P835; B305D; B854; CASB618 (as described in WO00/53748); CASB7439 (HASH-2, also described in WO01/62778); C1491; C1584; and C1585. In one embodiment, of the present invention, the antigen or MAGE antigen is MAGE-A3.

In one embodiment, the antigen may comprise or consist of P501S (also known as prostein). The P501S antigen may be a recombinant protein that combines most of the P501S protein with a bacterial fusion protein comprising the C terminal part of protein LytA of Streptococcus pneumoniae in which the P2 universal T helper peptide of tetanus toxoid has been inserted, ie. a fusion comprising CLytA-P2-CLyta (the “CPC” fusion partner), as described in WO03/104272.

In one embodiment, the antigen may comprise or consist of WT-1 expressed by the Wilm's tumour gene, or its N-terminal fragment WT-1F comprising about or approximately amino acids 1-249.

In one embodiment, the antigen may comprise or consist of a Her-2/neu antigen. For example, the Her-2/neu antigen may comprise or consist of one of the following fusion proteins which are described in WO00/44899: a “HER-2/neu ECD-ICD fusion protein,” also referred to as “ECD-ICD” or “ECD-ICD fusion protein,” which refers to a fusion protein (or fragments thereof) comprising the extracellular domain (or fragments thereof) and the intracellular domain (or fragments thereof) of the HER-2/neu protein. In one embodiment, this ECD-ICD fusion protein does not include a substantial portion of the HER-2/neu transmembrane domain, or does not include any of the HER-2/neu transmembrane domain.

In a further embodiment, the Her-2/neu antigen may comprise or consist of “HER-2/neu ECD-PD fusion protein,” also referred to as “ECD-PD” or “ECD-PD fusion protein,” or the “HER-2/neu ECD-ΔPD fusion protein,” also referred to as “ECD-ΔPD” or “ECD-ΔPD fusion protein,” which refers to fusion proteins (or fragments thereof) comprising the extracellular domain (or fragments thereof) and phosphorylation domain (or fragments thereof, e.g., ΔPD) of the HER-2/neu protein. In one embodiment, the ECD-PD and ECD-ΔPD fusion proteins do not include a substantial portion of the HER-2/neu transmembrane domain, or does not include any of the HER-2/neu transmembrane domain.

In an embodiment of the invention in which the antigen is a MAGE antigen, the antigen may be full length MAGE, substantially full-length MAGE or a fragment of MAGE, for example a peptide of MAGE. A substantially full-length antigen of MAGE that may be used in the present invention is amino acids 3-314 of MAGE-A3 (312 amino acids in total), or other fragments of MAGE antigens in which between 1 and 10 amino acids are deleted from the N-terminus and/or C-terminus of the MAGE protein sequence.

Examples of MAGE peptides that may be used in the present invention include the following MAGE-A3 peptides:

| SEQ ID NO | Peptide sequence |
|---|---|
| SEQ ID NO: 40 | FLWGPRALV |
| SEQ ID NO: 41 | EVDPIGHLY |
| SEQ ID NO: 42 | MEVDPIGHLY |
| SEQ ID NO: 43 | VHFLLLKYRA |
| SEQ ID NO: 44 | LVHFLLLKYR |
| SEQ ID NO: 45 | LKYRAREPVT |
| SEQ ID NO: 46 | ACYEFLWGPRALVETS |
| SEQ ID NO: 47 | TQHFVQENYLEY |

### Fusion Proteins

In one embodiment, the antigen may be linked to a fusion partner protein.

The antigen may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner protein may assist in providing T helper epitopes (immunological fusion partner protein), preferably T helper epitopes recognised by humans, and/or assist in expressing the protein (expression enhancer protein) at higher yields than the native recombinant protein. In one embodiment, the fusion partner protein may be both an immunological fusion partner protein and expression enhancing partner protein.

In one embodiment of the invention, the immunological fusion partner protein that may be used is derived from protein D, a surface protein of the gram-negative bacterium, Haemophilus influenza B (WO91/18926) or a derivative thereof. The protein D derivative may comprise the first ⅓ of the protein, or approximately the first ⅓ of the protein. In one embodiment, the first N-terminal 109 residues of protein D may be used as a fusion partner to provide an antigen with additional exogenous T-cell epitopes and increase expression level in E. coli (thus acting also as an expression enhancer). In an alternative embodiment, the protein D derivative may comprise the first N-terminal 100-110 amino acids or approximately the first N-terminal 100-110 amino acids. In one embodiment, the protein D or derivative thereof may be lipidated and lipoprotein D may be used: the lipid tail may ensure optimal presentation of the antigen to antigen presenting cells. In an alternative embodiment, the protein D or derivative thereof is not lipidated. The “secretion sequence” or “signal sequence” of protein D, refers to approximately amino acids 1 to 16, 17, 18 or 19 of the naturally occurring protein. In one embodiment, the secretion or signal sequence of protein D refers to the N-terminal 19 amino acids of protein D. In one embodiment, the secretion or signal sequence is included at the N-terminus of the protein D fusion partner. As used herein, the “first third”, “first 109 amino acids” and “first N-terminal 100-110 amino acids” refer to the amino acids of the protein D sequence immediately following the secretion or signal sequence. Amino acids 2-K and 3-L of the signal sequence may optionally be substituted with the amino acids 2-M and 3-D.

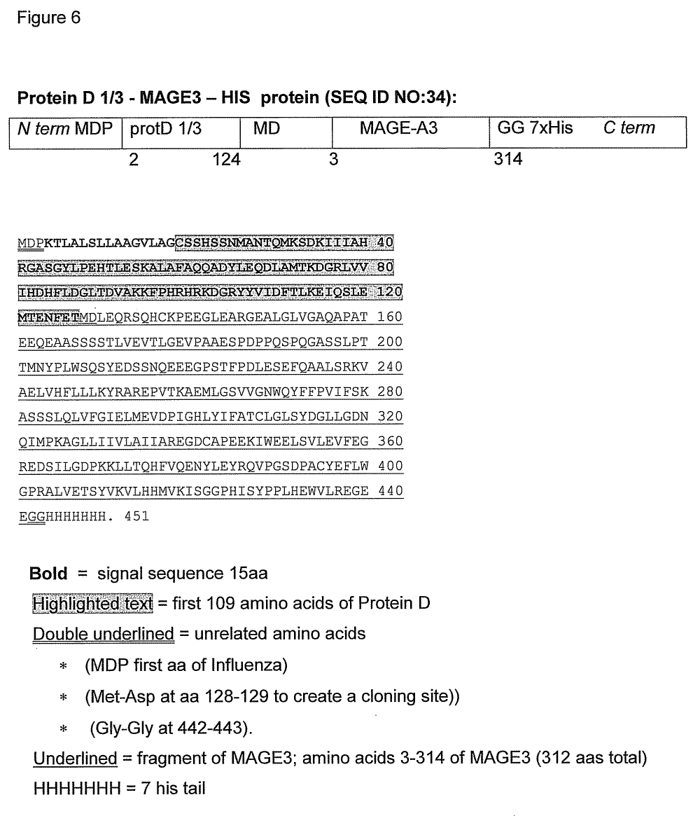

In one embodiment, the fusion protein may be Protein D-MAGE-A3-His, a 432-amino-acid-residue fusion protein. This fusion protein comprises the signal sequence of protein D, amino acids 1 to 109 of Protein D, 312 amino acids from the MAGE-A3 protein (amino acids 3-314), a spacer and a polyhistidine tail (His) that may facilitate the purification of the fusion protein during the production process, for example:
i) An 18-residue signal sequence and the first N-terminal 109 residues of protein D;
ii) Two unrelated residues (methionine and aspartic acid);
iii) Residues 3-314 of the native MAGE-3 protein;
iv) Two glycine residues functioning as a hinge region
v) seven Histidine residues.
   The amino acid sequence for this molecule is shown in FIG. 6 (SEQ ID NO:34). This antigen and those summarised below are described in more detail in WO99/40188.

In another embodiment the immunological fusion partner protein may be the protein known as LytA or a protein derived therefrom. LytA is derived from Streptococcus pneumoniae which synthesise an N-acetyl-L-alanine amidase, amidase LytA, (coded by the LytA gene (Gene, 43 (1986) page 265-272)) an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Biotechnology: 10, (1992) page 795-798). In one embodiment, the C terminal portion of the molecule may be used. The embodiment may utilise the repeat portion of the LytA molecule found in the C terminal end starting at residue 178. In one embodiment, the LytA portion may incorporate residues 188-305.

Other fusion partners include the non-structural protein from influenzae virus, NS1 (hemagglutinin). In one embodiment, the N terminal 81 amino acids of NS1 are utilised, although different fragments may be used provided they include T-helper epitopes.

In one embodiment of the present invention, the MAGE-A3 antigen described herein may comprise a derivatised free thiol. Such antigens have been described in WO99/40188. In particular carboxyamidated or carboxymethylated derivatives may be used.

In a further embodiment of the present invention, the composition or immunogenic composition may comprise a nucleic acid encoding an antigen or derivative as described herein, for example a nucleic acid-based vaccine or immunogenic composition may be used. This may comprise a nucleic acid molecule encoding an antigen or fusion protein as described herein. Nucleic acid sequences may be administered directly, as part of particle-mediated delivery (PMED), and/or may be inserted into a suitable expression vector and used for DNA/RNA vaccination. Vectors may include for example poxvirus, adenovirus, alphavirus and listeria.

Conventional recombinant techniques for obtaining nucleic acid sequences, and production of expression vectors of are described in Maniatis et al., Molecular Cloning—A Laboratory Manual; Cold Spring Harbor, 1982-1989.

Compositions, vaccines, antigens or components thereof as described herein for use in the present invention are provided either in a liquid form or in a lyophilised form.

Each human dose may comprise 1 to 1000 μg of protein, for example 30-300 μg.

### Adjuvants

The compositions described herein may further comprise an adjuvant, and/or an immunostimulatory cytokine or chemokine.

Adjuvants that may be used in the present invention include Merck Adjuvant 65 (Merck and Company, Inc., Rahway, N.J.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In one embodiment, the adjuvants may include, for example, a combination of monophosphoryl lipid A, such as 3-de-O-acylated monophosphoryl lipid A (3D-MPL) together with an aluminium salt.

In place of 3D-MPL, other toll like receptor 4 (TLR4) ligands such as aminoalkyl glucosaminide phosphates (WO 98/50399, WO 01/34617 and WO 03/065806) may be used.

In one embodiment, the adjuvant may include a TLR9 agonist such as an immunostimulatory oligonucleotide comprising unmethylated CpG, for example:

| | |
|---|---|
| SEQ ID NO: 35 | (CpG 1826) |
| SEQ ID NO: 36 | (CpG 1758) |
| SEQ ID NO: 37 | (CpG 2006) |
| SEQ ID NO: 38 | (CpG 7909) |
| SEQ ID NO: 39 | (CpG 1668) |

In one embodiment of the present invention, the adjuvant comprises the combination of a CpG-containing oligonucleotide and a saponin derivative, for example the combination of CpG and QS21 (WO 00/09159 and WO 00/62800).

The adjuvant formulation may additionally comprise an oil in water emulsion and/or tocopherol.

In one embodiment, the adjuvant comprises a saponin, for example QS21 (Aquila Biopharmaceuticals Inc., Framingham, Mass.), that may be used alone or in combination with other adjuvants. In one embodiment, the adjuvant comprises the combination of a monophosphoryl lipid A and saponin derivative, such as the combination of QS21 and 3D-MPL (WO 94/00153), or a composition where the QS21 is quenched with cholesterol (WO 96/33739).

In one embodiment, the adjuvant components are provided in an oil-in-water emulsion and tocopherol. In one embodiment, the adjuvant formulation comprises QS21, 3D-MPL and tocopherol in an oil-in-water emulsion (WO 95/17210).

In another embodiment, the adjuvants may be formulated in a liposomal composition.

In an embodiment, the adjuvant system comprises a CpG oligonucleotide, 3D MPL and QS21 either presented in a liposomal formulation or an oil in water emulsion (WO 95/17210).

Generally, it is expected that each human dose will comprise 0.1-1000 μg of antigen, preferably 0.1-500 μg, preferably 0.1-100 μg, most preferably 0.1 to 50 μg. An optimal amount can be ascertained by standard studies.

Following initial administration or vaccination, subjects may receive one or several booster administrations or immunisations adequately spaced.

Other adjuvants that may be used include Montanide ISA 720 (Seppic, France), SAF (Chiron, Calif., United States), ISCOMS (CSL), MF-59 (Chiron), Ribi Detox, RC-529 and other aminoalkyl glucosaminide 4-phosphates (AGPs) (GSK, Hamilton, Mont.).

In one embodiment, the adjuvant may comprise one or more of 3D-MPL, QS21 and an immunostimulatory CpG oligonucleotide. In an embodiment all three immunostimulants are present. In another embodiment 3D-MPL and QS21 are presented in an oil in water emulsion, and in the absence of a CpG oligonucleotide.

A composition for use in the method of the present invention may comprise a pharmaceutical composition comprising tumour associated antigen as described herein, or a fusion protein, in a pharmaceutically acceptable excipient.

### EXPERIMENTAL EXAMPLES

### 1. MAGE008 Mage Melanoma Clinical Trial

1.1. General Background Information

In this phase II trial, the recMAGE-A3 protein (a recombinant fusion protein comprising protein-D-MAGE-A3) was combined with two different immunological adjuvants: either AS02B (QS21 and MPL in an oil-in water emulsion) or AS15 (QS21, MPL and CpG7909 in a liposomal formulation). The objectives were to discriminate between the adjuvants in terms of safety profile, clinical response and immunological response.

1.2. Study Overview:

1.2.1. Design

The MAGE008 trial was:

open

randomized

two-arm (AS02B vs. AS15)

With 68 patients in total.

As described above, the recMAGE-A3 protein was combined with either AS02B or AS15 adjuvant system.

1.2.2. Patient Population

The recMAGE-A3 protein was administered to patients with MAGE-A3 positive, progressive metastatic melanoma with regional or distant skin and/or lymph-node lesions (unresectable stage III and stage IV M1a). The expression of the MAGE-A3 gene by the tumour was assessed by quantitative PCR. The selected patients did not receive previous treatment for melanoma (recMAGE-A3 is given as first-line treatment) and had no visceral disease.

1.2.3. Schedule of Immunization

1.2.3.1. Method of Treatment Schedules

The immunization schedule followed in the MAGE008 clinical trial was:

| | |
|---|---|
| Cycle 1: | 6 vaccinations at intervals of 2 weeks |
| | (Weeks 1, 3, 5, 7, 9, 11) |
| Cycle 2: | 6 vaccinations at intervals of 3 weeks |
| | (Weeks 15, 18, 21, 24, 27, 30) |
| Cycle 3: | 4 vaccinations at intervals of 6 weeks |
| | (Weeks 34, 40, 46, 52) |
| Long Term Treatment: | 4 vaccinations at intervals of 3 months, |
| | for example followed by |
| | 4 vaccinations at intervals of 6 months |

For both of the above treatment regimes additional vaccinations may be given after treatment, as required.

In order to screen potential participants in the above clinical trial we received biopsies of the tumour (both prior to any immunization and after immunization, if applicable, as relapses, as frozen tumour samples. Relapse samples were not included in the analysis to generate the gene list and predictive models of the invention including in the examples). From these samples, RNA was extracted for quantitative PCR. The purified RNA was also suitable for microarray analysis. We therefore analyzed the tumour samples by microarrays. The goal was to identify in pre-vaccination biopsies a set of genes associated with the clinical response and to develop a mathematical model that would predict patient clinical outcome, so that patients likely to benefit from this antigen-specific cancer immunotherapeutic are properly identified and selected. This gene profiling analysis has been performed only on biopsies from patients who signed the informed consent for microarray analysis.

### 2. Materials and Methods

2.1. Tumour Specimens and RNA Purification

86 tumour biopsies taken from 68 patients (precisely 70 primary tumour biopsies, pre-vaccination type, 17 relapse biopsies, after vaccination type) were used from the Mage008 Mage-3 melanoma clinical trial. These were fresh frozen preserved in the RNA stabilizing solution RNAlater.

Total RNA was purified using the Tripure method (Roche Cat. No. 1 667 165). The provided protocol was followed subsequently by the use of an RNeasy Mini kit—clean-up protocol with DNAse treatment (Qiagen Cat. No. 74106). RNA from the samples whose melanin content was high (determined by visual inspection) was further treated using CsCl centrifugation.

The Quality of the RNA was assessed by use of the Agilent bioanalyser. Quantification of RNA was initially completed using optical density at 260 nm and Quant-IT RiboGreen RNA assay kit (Invitrogen—Molecular probes R11490).

2.2. RNA Labelling and Amplification for Microarray Analysis

Due to the small biopsy size received during the clinical study, an amplification method was used in conjunction with the labelling of the RNA for microarray analysis: the Nugen 3′ ovation biotin kit (Labelling of 50 ng of RNA—Ovation biotin system Cat; 2300-12, 2300-60). A starting input of 50 ng of total RNA was used.

2.3. Microarray Chips, Hybridizations and Scanning

The Affymetrix HG-U133.Plus 2.0 gene chips were hybridized, washed and scanned according to the standard Affymetrix protocols. Some RNAs were replicated on arrays, making 96 the total number of available hybridizations for subsequent analysis.

2.4. Sample Normalization

The fluorescent scanned data (a.k.a the .CEL files) was processed and normalized using a R 2.4.1 implementation of the GCRMA algorithm (Jean (ZHIJIN) Wu and Rafael Irizarry with contributions from James MacDonald Jeff Gentry (2005). gcrma: Background Adjustment Using Sequence Information. R package version 2.6.0). All 96 hybridizations were included in the normalization process, making the individual hybridizations and associated patient gene expression profile comparable between each other (Wu et al., 2004).

2.5. Non-Specific Filtering

The probe sets (PS) of normalized hybridization samples are filtered independently of the outcome associated to each sample. The objective of this non-specific filtering is to get rid of genes showing roughly constant expression across samples as they tend to provide little discrimination power (Heidebreck et al., 2004).

Only the most variant quartile of features are kept, the other PS values being simply discarded. This step typically reduces the PS size from 54,613 down to about 13,650.

2.6. Feature Normalization

The filtered probe sets (PS) of each sample are subsequently normalized according to an IQR-Normalization procedure. The goal of this second normalization step is to make more similar the genes sharing a common expression pattern throughout the data but in different ranges of absolute expression values.

The IQR-Normalization for each PS individual patient expression value is calculated as follows. A PS-specific median is subtracted from the PS value. The median-centered expression value is then weighted by a PS-specific Inter Quartile Range (IQR) (Weisstein) divided by 1.35. The 1.35 scaling factor makes sure that the IQR normalization becomes equivalent to a Z-score normalization whenever the data is normally distributed.

The PS-specific medians and IQRs involved in the IQR-Normalization calculation are those calculated from the training set, that is the set of samples used to estimate the predictive model (see evaluation protocol, section 2.8).

2.7. Feature Selection and Supervised Classification

The objective of feature selection is to find a small set of genes, or reporters, the expressions of which are highly predictive of the patient clinical outcome. The outcome of interest is whether the patient will be a responder or non-responder to the MAGE-A3 ASCI treatment. Responder patients are those presenting a clinical benefit, i.e. either a complete response, a partial response, a mixed response or a stable disease for four months.

Hence, feature selection is intrinsically related here with a binary classification problem. Hard-margin linear support vector machines (SVM) (Boser et al., 1992; Schölkopf and Smola, 2002) were used to predict the MageA3 patient clinical outcomes.

We consider two feature selection algorithms belonging to the class of multivariate embedded methods (Guyon et al., 2006). Multivariate methods measure how well a set of features are jointly discriminative rather than considering them individually. Hence the reporter selection relies on the potential dependencies between different gene expressions. Embedded methods aim at incorporating feature selection directly in the estimation of the predictive model, here a linear SVM. We combined here two embedded methods: RFE (Guyon et al., 2003) and L2AROM (Weston et al., 2003).

Both selection methods are iteratively discarding features. For RFE, the PS list is reduced in two iterations from 13,650 features down to 8192 and finally 4,096 features which form the so-called RFE-list. For L2AROM, the number of selected features is not fixed by the user but results from the optimization procedure itself. L2AROM was started with 13,650 features and stopped before falling below 4,096 features. The RFE selection criterion was then applied to this list to obtain the so-called L2AROM-list of exactly 4,096 features.

The two selection methods described above were repeatedly applied on 30 bootstrap resampling of the original data set. For each such resampling, a RFE-list and a L2AROM-list (each one containing 4,096 features) are constructed, forming a total of 60 reporter lists. A final selection is obtained by intersecting the 60 reporter lists resulting in a list of only 33 PS. The advantage of such a 33-consensus list is to reduce the variability of the reporter list obtained according to a particular sampling of the data set. It also drastically reduces the final number of reporters without the need for fixing arbitrarily this number. The final predictive model is a hard-margin linear SVM estimated on the whole data set reduced to the 33 consensus features.

2.8. Evaluation Protocol

The performance measures used to evaluate the quality of the feature selection and classification methods are the sensitivity, the specificity, the balanced classification rate (BCR), which is the arithmetic average between sensitivity and specificity, and the positive predictive value, also known as clinical efficacy in the present context.

We rely here on the Bootstrap 0.632 protocol due to Efron (Efron, 1983). It is known to reduce the often large variance of cross-validation procedures. The Bootstrap 0.632+ is an additional refinement which further reduces the positive bias observed with some classification rules (Efron and Tibshirani, 1997). The specific version of the Bootstrap 0.632 protocol is explicit in the reported results. For each resampled data set, forming a bag representing on average 63.2% of the original data set, a hard-margin linear SVMs is estimated from the 33 feature values measured for each patient in the bag. The final performance is computed over 1,000 bootstrap resamplings.

### 3. Results

All results are related to a set of 62 primary biopsies taken from 61 patients for whom the clinical response status was known at the time of these experiments. The patient samples were labelled accordingly as responders or non-responders. 22 samples were labelled as responders and the remaining 40 as non-responders.

3.1. 33 PS Reporter List

Table 1 details the 33 reporter list obtained using the consensus methodology described above.

3.2. Bootstrap 0.632+ Performances

The performances of hard-margin linear SVMs built on the 33 reporter list is summarized in Table 2. These performances were computed with the Bootstrap 0.632+ protocol from 1,000 independent resamplings of the data-set.

Those results illustrate the high predictive power of the models. In summary, a 33 probe-set list correctly predicts 91% of the patient clinical response with hard-margin linear Support Vector Machines.

3.3. Permutation Tests

A permutation test was performed to check whether the 33 reporter list may offer high predictive power without relation to the clinical outcome. The testing procedure was designed as follows. The outcome labels were randomly permuted 1,000 times. For each permutation, a Bootstrap 0.632 protocol was applied with 30 resamplings. For each permutation and each resampling, a hard-margin linear SVM was estimated resulting in a total of 30,000 so-called random models.

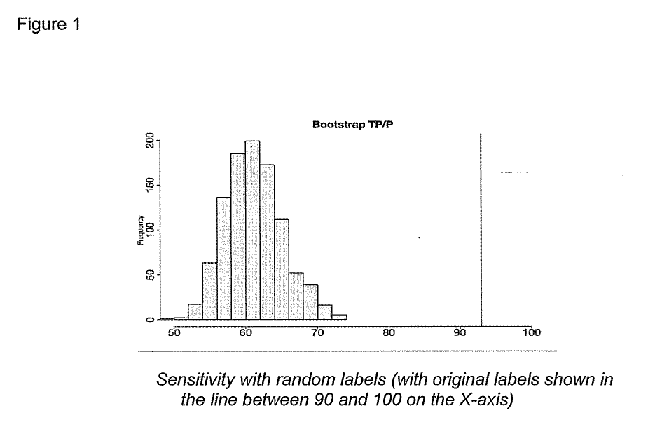

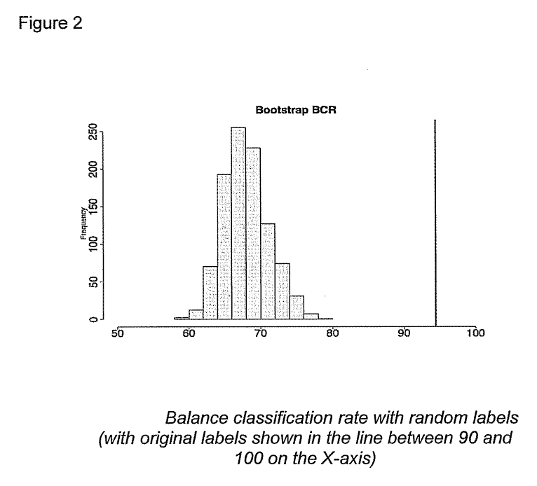

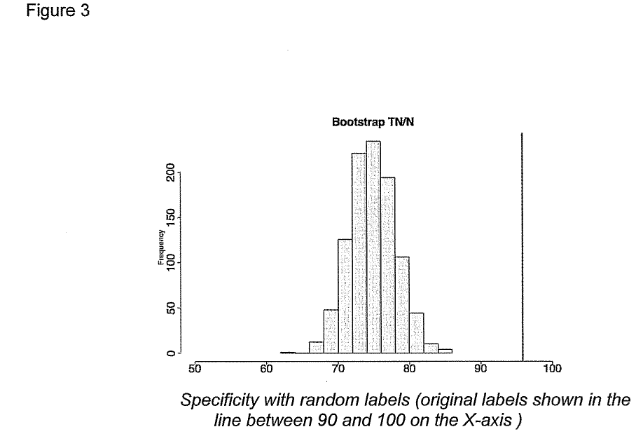

FIGS. 1, 2 and 3 presents the histograms of the BCR, sensitivity and specificity of models built from random labels. The B-632 performances (with 30 resamplings) of the reference models built from the original labels are reported in the line between 90 and 100 on the X-axis in these figures. None of the 1,000 permutations results in better performances than the reference models.

In summary, the hypothesis that a hard-margin linear SVM based on the 33 reporter list may better predict a random outcome than the actual clinical outcome is rejected with a p-value<0.001.

3.4. Impact of the 33 PS Feature List Based SVM Predictive Model on Treatment Failure

The 33 PS reporter list or gene expression signature enables differentiation of patients who respond to the administration of the MAGE-A3 ASCI and patients who do not. The time to treatment failure data from this study was analyzed according to the clinical outcome prediction given the 33 PS gene expression signature and associated SVM classifier prior to ASCI administration.

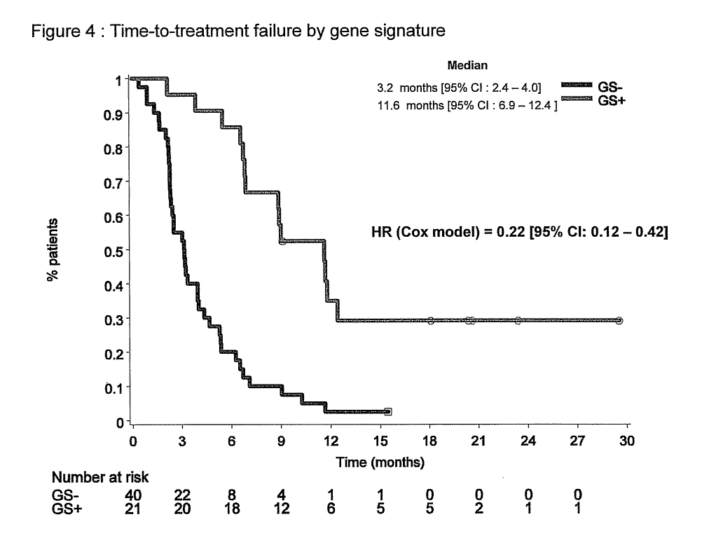

A Kaplan-Meier curve is presented in FIG. 4. Increased time-to-treatment failure is observed in the gene signature (GS) positive population (i.e. the population of patients predicted by the gene signature as responder to ASCI treatment). The median time-to-treatment failure is 11.6 months (95% CI 6.9-12.4) in the GS positive population whereas it is 3.2 months (95% CI 2.4-4.0) in the GS negative population of patients. The Hazard Ratio (HR) is 0.22 (95% CI 012-0.42), i.e. the risk of treatment failure is reduced by 78% in the GS positive population of patients.

### 4. Conclusion

Applying the 33 PS predictive model to select the patients for treatment increases the MageA3 ASCI clinical efficacy. Expected clinical efficacy, as estimated by the Positive Predictive Value (Table 2) is 91%. As a comparison clinical efficacy without the 33 PS predictive model input for the selection of patients is 35%.

Applying the 33 PS predictive model to select the patients for treatment also reduces the risk of MageA3 ASCI treatment failure by 78%.

### 5. Implementation of the 33 Probe-Set Predictive Model, Predicting the Clinical Outcome of Further Patient Biopsies

5.1. Sample Normalization

The independent patient-derived samples one would like to analyse with the 33 PS feature list based SVM model, would first be normalized following the normalisation scheme that was used and described in the Sample normalization section of the Materials and Methods.

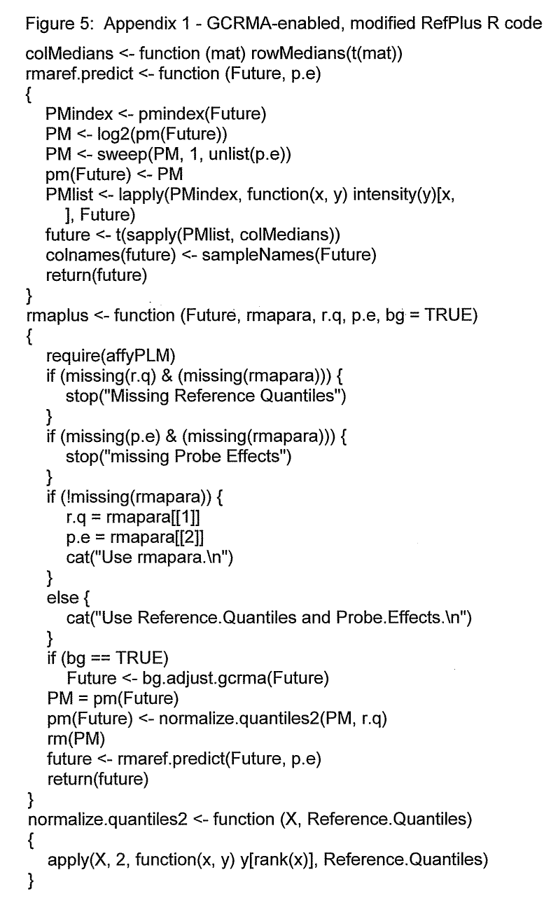

To apply the GCRMA normalisation scheme under the normalisation parameters used in the previous sections, one would use the R code produced in Appendix 1 (FIG. 5) in a R 2.6.0 session. Bioconductor release 2.1 packages (http://bioconductor.org) are available.

The Appendix 1 (FIG. 5) code chunk is a modification of the code contained in the RefPlus R package (Harbron et al., 2007), available in Bioconductor. The RefPlus code is modified to perform a GCRMA normalization of a given sample hybridization, taking into account normalization parameters calculated from a reference data set. The reference dataset is the data set described in the previous sections. RefPlus is initially designed for reference data set normalization, but uses the RMA algorithm rather than the GCRMA. The only difference between RMA and GCRMA lies in the background correction step. RefPlus was enabled to perform GCRMA background correction by replacing the bg.correct.rma R function embedded in the rmaplus R function by the bg.adjust.gcrma R function. The RefPlus code modification was done in October 2007 and is available from GlaxoSmithKline.

To normalize a sample with GCRMA-enabled, modified RefPlus code of Appendix 1 (FIG. 5), one would have to call the GCRMA background correction enabled-rmaplus function, with, as parameters, besides the data to normalize (of class AffyBatch), the reference quantiles (r.q option) and probe effect (p.e option) that are calculated on the reference data set. The reference quantiles and probe effects are contained in the rq.txt and pe.txt files, available from the Head of Corporate Intellectual Property at GSK. These files have also been submitted to the USPTO on a Compact Disc as referenced above. These files are also available as pgp encrypted files at http://www.cordina.org.uk/pe.gpg and http://www.cordina.org.uk/rq.gpg so that it is possible to access them on the filing date of the application. The files can be decrypted using any standard PGP software (for example as available at http://www.gnupg.org). The key to decrypt these files is as follows and the passphrase for the key is gsk (lower case)

-----BEGIN PGP PRIVATE KEY BLOCK-----
Version: GnuPG v1.4.9 (MingW32)

IQHhBEqqDiERBACqgNnyIFS9lilA5a3mx4oab0zhsO5ZhiWTbYnaYJTPmGefqN8bcyc6
BbRQ5oUM96Ch4IeA2ohrwBfHEZRU3sAbl3XTGfd6ZCGdFLPIOuOlJZmherw3WGlpv1
qDDghJVmYwEm/3TeFq/ZURX4tDeCmQZq0xgNFY9gnbSMlQ/5RDwwCgoEpxYEPTU
nRsZHX4xWLiWnVuLgsD/iCPGiDLkHJ3Ghe9FUfiJhgcYN7I9MZVD3EmE1CcP0GUx9
CrjxwMzNCoIBoYM9MJkqup651KyBr2CgqBomfnmlUmepktkpUIq8SGva8P/Iv8uEy/Xek
QdTVJWmnofVCkfVLPLvlfvQ/zn/r+4H2+oiC6wFhbco15xBQggDTefCmnA/46LWRtZj9B
0g/beOYBhoKaRcmefXQqXUyZ5wgvLfph5mpJz4FlcwQW4c9SHMnOVM4klwGRq0W0
q8fF8AiJcLS4T1Fm5ufS+GV4eHmWigjFeDs5E8ZsIHDehOTV+h9yJRRqmDDqBIOsZk
mBFDP/4eboHhojlOlmomeM+JvsUegBaf4DAwI1sR9cDEPCwGA871NLeReTmgnIzTY9
X33cK0i789HfvFOxGczPFm7VEJtjG7cfU3l/kAxNcrfdGZFdlrQgR1NLIDxyb2JlcnQuc3RI
cGhlbkBvbHN3YW5nLmNvbT6IYAQTEQIAIAUCSqoOIQIbAwYLCQgHAwIEFQIIAwQW
AgMBAh4BAheAAAoJELi/cfygwol3qP8An2jV0hVwuu/rM9XSc5ONj9YWiM/fAKCQNFm
NgsaZW9s8OfEgoyN3tV8gep0CYwRKqg4hEAgAvoDU1FPP58U4cYhcz/B1rrpI0vjeCH
qKQYx81TVk4ibg8iSD5+VH2Is0wOODBG15MEm+HKKD5MDU4kMI8X78WuSYsxPIE
WuJXnMTLcmUh/42uOugHksZnIh8QEMlSZGoVo86veXwViHE3IOYe2VmY/EcRlZ1RK
CJQgW1JZsZ34r0CTh1kC4daXDEKcw5CHGefGVIRZnANwke+M+/AXw+ehZLXZ2m0
Dh67yPM5iRLKrXBdjlwlDtQB6w/334vkZbv8AvgFbX0WWFQtOOma9MsAMqtMRxfvwv
WRK9pu1ab6/uCIPyb9htR9YPhJFpCrMrMoD6Bc971AxL4RhONwvTF2wADBQf9HP/pn
GNosJQzStJ0gs/YPyCIj2U46A6nN/VdhPacI7BQzGAbGcE07bJ5qlJYkVI2XrorLQ6sileB
C7uMne3p0ALQ6dMNb2SHXiswsvnwrhGXTBcZgMdn55GZhJMh1ibAC7HiS7iPj+fHLN
yfZ9aQG8RqKdgfJFWe3SVIVwmXkGxNPSzz1FsW/D26a779ncCPhfK6VW4Xx6lcn6yis
rbNiK4mEC4EtRTlolUlE8slfarSOxlWEgo9I1NUTcTYY/u6YNKRWddjFZtls4HQBEpDaR
KQzCsaZeKwcfRtLfFk13HL1+Va4Rcr69DZ39tUyvmeEEEjWxh99LVCUt0aseWlsf4DAw
I1sR9cDEPCwGCK6NDETt59802regCUqKWfM1pYgc3fuLh4WEBXz6e+60LZJeKIzi+iV
I1DBcmp+7dVd+dsD8D56MkyEEMP7NHakU70pdKzPYh3iEkEGBECAAkFAkqqDiECG
wwACgkQuL9x/KDCiXekBQCeMBLDjs2QAYJiXwEUiaPW4hESIVgAn3o9JfPDNNwpup
dGuy9OTEYmphL+=U2WR

-----END PGP PRIVATE KEY BLOCK-----

5.2 Feature Normalization and Outcome Prediction

The following R code chunk in a R 2.4.1 session predicts the clinical response of further samples.

```
norm<-function(x,ms,sds){
    for(i in 1:length(x)){
        x[i]=(x[i]−ms[i])/sds[i]
    }
    return(x)
}
mySVM<-function(data,weights,rho){
    predictions=c( );
    for(i in 1:dim(data)[1]){
        if((data[i,]%*%weights)−rho>0){
            predictions=c(predictions,1)
        }
        else{
            predictions=c(predictions,−1)
        }
    }
    return(predictions)
}
normTest=norm(test,medians,iqrs)
predictions=mySVM(normTest,w,rho)
```

where
test is an object of class matrix of 33 columns containing the 33 features of probe sets corresponding to indexes available in Table 3 (column 1) as columns and expression data (GCRMA processed) of the further samples to predict as rows.
medians is an object of class matrix containing the 33 values of Table 3 (column 2)
iqrs is an object of class matrix containing the 33 values of Table 3 (column 3)
w is an object of class matrix containing the 33 values of Table 3 (columns 4)
rho is a numeric variable containing the value 0.7596883

TABLE 1
33 Probe Set Report List

| | Gene | | Immune |
|---|---|---|---|
| Probe Set | symbol | Gene Name | related |
| 1555852_at | — | proteasome (prosome, macropain) subunit, | |
| | | beta type, 9 (large multifunctional peptidase 2) | |
| 1557116_at | APOL6 | apolipoprotein L, 6 | |
| 200986_at | SERPING1 | serpin peptidase inhibitor, clade G (C1 | Yes |
| | | inhibitor), member 1, (angioedema, | |
| | | hereditary) | |
| 201474_s_at | ITGA3 | integrin, alpha 3 (antigen CD49C, alpha 3 | |
| | | subunit of VLA-3 receptor) | |
| 202307_s_at | TAP1 | transporter 1, ATP-binding cassette, sub- | |
| | | family B (MDR/TAP) | |
| 202531_at | IRF1 | interferon regulatory factor 1 | |
| 202659_at | PSMB10 | proteasome (prosome, macropain) subunit, | Yes |
| | | beta type, 10 | |
| 202768_at | FOSB | FBJ murine osteosarcoma viral oncogene | |
| | | homolog B | |
| 204116_at | IL2RG | interleukin 2 receptor, gamma (severe | Yes |
| | | combined immunodeficiency) | |
| 205499_at | SRPX2 | sushi-repeat-containing protein, X-linked 2 | |
| 205814_at | GRM3 | glutamate receptor, metabotropic 3 | |
| 205890_s_at | GABBR1 | ubiquitin D | |
| | /// UBD | | |
| 208306_x_at | HLA-DRB1 | major histocompatibility complex, class II, DR | Yes |
| | | beta 4 | |
| 208729_x_at | HLA-B | major histocompatibility complex, class I, B | Yes |
| 208812_x_at | HLA-C | major histocompatibility complex, class I, C | Yes |
| 209040_s_at | PSMB8 | proteasome (prosome, macropain) subunit, | Yes |
| | | beta type, 8 (large multifunctional peptidase 7) | |
| 210306_at | L3MBTL | l(3)mbt-like (Drosophila) | |
| 211911_x_at | FAM20B /// | major histocompatibility complex, class I, B | Yes |
| | HLA-B /// | | |
| | HLA-C /// | | |
| | MICA /// | | |
| | MICB /// | | |
| | XXbac- | | |
| | BPG181B23.1 | | |
| 214617_at | PRF1 | perforin 1 (pore forming protein) | Yes |
| 216920_s_at | TARP /// | TCR gamma alternate reading frame protein | Yes |
| | TRGC2 | | |
| 218553_s_at | KCTD15 | potassium channel tetramerisation domain | |
| | | containing 15 | |
| 219505_at | CECR1 | cat eye syndrome chromosome region, | |
| | | candidate 1 | |
| 221875_x_at | HLA-F | major histocompatibility complex, class I, F | Yes |
| 223264_at | MESDC1 | mesoderm development candidate 1 | |
| 224225_s_at | ETV7 | ets variant gene 7 (TEL2 oncogene) | |
| 225502_at | DOCK8 | dedicator of cytokinesis 8 | |
| 225973_at | TAP2 | transporter 2, ATP-binding cassette, sub- | Yes |
| | | family B (MDR/TAP) | |
| 228362_s_at | FAM26F | family with sequence similarity 26, member F | |
| 229391_s_at | FAM26F | family with sequence similarity 26, member F | |
| 232035_at | HIST1H4A/ | histone cluster 1, H4h | |
| | HIST1H4B/ | | |
| | HIST1H4C/ | | |
| | HIST1H4D/ | | |
| | HIST1H4E/ | | |
| | HIST1H4F/ | | |
| | HIST1H4H/ | | |
| | HIST1H4I/ | | |
| | HIST1H4J/ | | |
| | HIST1H4K/ | | |
| | HIST1H4L/ | | |
| | HIST2H4A/ | | |
| | HIST2H4B/ | | |
| | HIST4H4 | | |
| 232615_at | — | phosphodiesterase 4D interacting protein | |
| | | (myomegalin) | |
| 242298_x_at | — | NA | |
| 244455_at | KCNT2 | potassium channel, subfamily T, member 2 | |

TABLE 2
Bootstrap .632+ performances

| | |
|---|---|
| Balance Classification Rate | 93% |
| Sensitivity | 91% |
| Specificity | 95% |
| Positive Predictive Value | 91% |

TABLE 3

| Probe Set | medians | iqrs | w |
|---|---|---|---|
| 202768_at | 4.2945 | 1.612407 | 0.356320091 |
| 242298_x_at | 5.6615 | 1.182778 | 0.286604321 |
| 232035_at | 6.0585 | 1.697037 | 0.033357645 |
| 205499_at | 4.0125 | 1.692407 | −0.475958034 |
| 225973_at | 7.486 | 1.468519 | −0.148430140 |
| 223264_at | 7.0175 | 1.508333 | −0.378014524 |
| 201474_s_at | 4.087 | 1.486481 | −0.231351682 |
| 200986_at | 10.027 | 1.323889 | 0.107823223 |
| 224225_s_at | 4.9895 | 1.969630 | 0.656910579 |
| 210306_at | 3.5015 | 1.216296 | −0.308518810 |
| 208729_x_at | 10.5335 | 1.492778 | −0.003427280 |
| 208812_x_at | 11.8695 | 1.138148 | 0.267930366 |
| 1555852_at | 6.8355 | 1.828148 | 0.130773917 |
| 229391_s_at | 9.13 | 2.667037 | 0.056165043 |
| 204116_at | 6.6715 | 3.532778 | 0.191608828 |
| 208306_x_at | 12.4225 | 1.033704 | −0.220028465 |
| 221875_x_at | 10.785 | 1.213333 | 0.002415460 |
| 219505_at | 10.6835 | 1.392222 | 0.217601264 |
| 205814_at | 4.623 | 1.198148 | 0.504196828 |
| 209040_s_at | 9.7995 | 1.048704 | −0.109093346 |
| 244455_at | 5.0795 | 1.951852 | −0.522628793 |
| 216920_s_at | 5.646 | 2.338704 | 0.134098709 |
| 225502_at | 7.145 | 1.576481 | 0.350031956 |
| 228362_s_at | 8.5405 | 2.752593 | 0.036450300 |
| 205890_s_at | 9.5615 | 3.263889 | 0.122724349 |
| 218553_s_at | 4.5175 | 1.057222 | −0.066717818 |
| 1557116_at | 10.611 | 1.417222 | 0.006990846 |
| 214617_at | 8.5575 | 3.273889 | 0.210212959 |
| 202307_s_at | 7.9595 | 1.777222 | −0.230227075 |
| 211911_x_at | 10.7935 | 1.509630 | 0.134222693 |
| 202531_at | 7.1365 | 2.112407 | −0.246445599 |
| 232615_at | 9.5505 | 1.004074 | 0.473009564 |
| 202659_at | 6.609 | 1.547037 | 0.095913418 |

TABLE 4
Probe Target Sequences

| Probe Set ID | Target Sequence Identifier |
|---|---|
| 1555852_at | SEQ ID NO: 1 |
| 1557116_at | SEQ ID NO: 2 |
| 200986_at | SEQ ID NO: 3 |
| 201474_s_at | SEQ ID NO: 4 |
| 202307_s_at | SEQ ID NO: 5 |
| 202531_at | SEQ ID NO: 6 |
| 202659_at | SEQ ID NO: 7 |
| 202768_at | SEQ ID NO: 8 |
| 204116_at | SEQ ID NO: 9 |
| 205499_at | SEQ ID NO: 10 |
| 205814_at | SEQ ID NO: 11 |
| 205890_s_at | SEQ ID NO: 12 |
| 208306_x_at | SEQ ID NO: 13 |
| 208729_x_at | SEQ ID NO: 14 |
| 208812_x_at | SEQ ID NO: 15 |
| 209040_s_at | SEQ ID NO: 16 |
| 210306_at | SEQ ID NO: 17 |
| 211911_x_at | SEQ ID NO: 18 |
| 214617_at | SEQ ID NO: 19 |
| 216920_s_at | SEQ ID NO: 20 |
| 218553_s_at | SEQ ID NO: 21 |
| 219505_at | SEQ ID NO: 22 |
| 221875_x_at | SEQ ID NO: 23 |
| 223264_at | SEQ ID NO: 24 |
| 224225_s_at | SEQ ID NO: 25 |
| 225502_at | SEQ ID NO: 26 |
| 225973_at | SEQ ID NO: 27 |
| 228362_s_at | SEQ ID NO: 28 |
| 229391_s_at | SEQ ID NO: 29 |
| 232035_at | SEQ ID NO: 30 |
| 232615_at | SEQ ID NO: 31 |
| 242298_x_at | SEQ ID NO: 32 |
| 244455_at | SEQ ID NO: 33 |

TABLE 5

| ProbeSet ID | Differential Expression in responders |
|---|---|
| 205499_at | Down-regulated |
| 223264_at | Down-regulated |
| 201474_s_at | Down-regulated |
| 210306_at | Down-regulated |
| 244455_at | Down-regulated |
| 218553_s_at | Down-regulated |
| 202768_at | Up-regulated |
| 242298_x_at | Up-regulated |
| 232035_at | Up-regulated |
| 225973_at | Up-regulated |
| 200986_at | Up-regulated |
| 224225_s_at | Up-regulated |
| 208729_x_at | Up-regulated |
| 208812_x_at | Up-regulated |
| 1555852_at | Up-regulated |
| 229391_s_at | Up-regulated |
| 204116_at | Up-regulated |
| 208306_x_at | Up-regulated |
| 221875_x_at | Up-regulated |
| 219505_at | Up-regulated |
| 205814_at | Up-regulated |
| 209040_s_at | Up-regulated |
| 216920_s_at | Up-regulated |
| 225502_at | Up-regulated |
| 228362_s_at | Up-regulated |
| 205890_s_at | Up-regulated |
| 1557116_at | Up-regulated |
| 214617_at | Up-regulated |
| 202307_s_at | Up-regulated |
| 211911_x_at | Up-regulated |
| 202531_at | Up-regulated |
| 232615_at | Up-regulated |
| 202659_at | Up-regulated |

### REFERENCES

Boser et al., “A training algorithm for optimal margin classifiers.”, Proceedings of the 5th Annual ACM Workshop on Computational Learning Theory, pages 144-152, Pittsburgh, Pa., USA, 1992.
Efron, “Estimating the Error Rate of a Prediction Rule: Improvement on Cross-Validation”, Journal of the American Statistics Association, Vol. 78, No. 382, pp. 316-331, 1983.
B. Efron and R. Tibshirani, “Improvements on Cross-Validation: the .632+Bootstrap Method”, Journal of the American Statistics Association, Vol. 92, No. 438, pp. 548-560, 1997.
Guyon et al., “Gene selection for cancer classification using support vector machines”, Machine Learning, Vol. 46, pp. 389-422, 2002.
Guyon et al., Editors. “Feature Extraction, Foundations and Applications”, Series Studies in Fuzziness and Soft Computing, Vol. 207, Springer, 2006.
Harbron et al., RefPlus: an R package extending the RMA algorithm. Bioinformatics 23:2493-2494 2007.
Heydebreck et al., “Differential Expression with the BioConductor Project”, BioConductor Project Working Paper, Paper 7, 2004 (http://www.bepress.com/bioconductor/paper7)
B. Schölkopf and A. Smola, “Learning with Kernels: Support Vector Machines, Regularization Optimization and Beyond”, MIT Press, Cambridge, Mass., 2002.
Eric W. Weisstein, “Interquartile Range.” From MathWorld—A Wolfram Web Resource. http://mathworld.wolfram.com/InterquartileRange.html
Weston et al., “Use of the zero norm with linear models and kernel methods”, The Journal of Machine Learning Research, Volume 3, pp. 1439-1461, 2003.
Wu et al.: A model-based background adjustment for oligonucleotide expression arrays. Journal of the American Statistical Association, 99:909-917, 2004.

1. A method of classifying a patient as a responder or non-responder comprising the steps of:
(a) determining the expression levels of one or more genes in a patient-derived sample, wherein the gene(s) are selected from Table 1;
(b) classifying the patient to either a responder or non-responder group based on the expression levels of (a).

2. A method of characterising a patient as a responder or non-responder to therapy comprising the steps:
(a) analysing a patient derived sample for differential expression of the gene products of one or more genes of Table 1, and
(b) characterising the patient from which the sample was derived as a responder or non-responder, based on the results of step (a),
wherein the characterisation step is optionally performed by reference or comparison to a standard.

3. A method according to claim 2, in which the standard is a patient-derived sample from a patient having a known clinical outcome.

4. A method according to claim 1, in which the one or more genes of Table 1 are at least 5, at least 10, at least 15, at least 20 and/or all the genes listed in Table 1 and/or any combination thereof.

5. A method according to claim 2, in which the one or more genes of Table 1 are at least 5, at least 10, at least 15, at least 20 and/or all the genes listed in Table 1 and/or any combination thereof.

6. A method according to claim 1, in which the one or more genes of Table 7 are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the genes of Table 1 and/or any combination thereof.

7. A method according to claim 2, in which the one or more genes of Table 7 are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all of the genes of Table 1 and/or any combination thereof.

8. A method according to claim 1, in which at least one gene product is upregulated.

9. A method according to claim 2, in which at least one gene product is upregulated.

10. A method according to claim 1, in which at least 80% of the gene products are upregulated.

11. A method according to claim 2, in which at least 80% of the gene products are upregulated.

12. A method according to claim 1, further comprising the step of determining whether the gene products are upregulated and/or downregulated as shown in Table 5.

13. A method according to claim 2, further comprising the step of determining whether the gene products are upregulated and/or downregulated as shown in Table 5.

14. A method according to claim 12, wherein a determination that the gene products are upregulated and/or downregulated as shown in Table 5 indicates a responder.

15. A method according to claim 13, wherein a determination that the gene products are upregulated and/or downregulated as shown in Table 5 indicates a responder.

16. A method as defined in claim 14, comprising the further step of identifying a patient as a responder, and selecting the patient for therapy.

17. A method as defined in claim 15, comprising the further step of identifying a patient as a responder, and selecting the patient for therapy.

18. A microarray comprising one or more polynucleotide probes complementary and hybridisable to a sequence of the gene product of at least one gene selected from the group of gene products listed in Table 1, in which the polynucleotide probes complementary and hybridisable to a sequence of the gene product of at least one gene selected from the group consisting of the gene products listed in Table 1 constitute at least 50% of the probes or probe sets on said microarray.

19. A method of treating a patient characterised as a responder comprising the steps of:
(a) determining the expression levels of one or more gene products in a patient-derived sample, wherein the gene products are selected from the group of gene products s set forth Table 1;
(b) classifying the patient to either a responder or non-responder group based on the expression levels of (a); and
(c) if the patient is classified as a responder, administering a composition comprising a tumour associated antigen to the patient.

20. A method of treating a patient characterised as a responder comprising the steps of:
(a) analysing a patient derived sample for differential expression of the gene products of one or more genes of Table 1;
(b) characterising the patient from which the sample was derived as a responder or non-responder, based on the results of step (a), wherein the characterisation step is optionally performed by reference or comparison to a standard; and
(c) if the patient is classified as a responder, administering a composition comprising a tumour associated antigen to the patient.
